(19) United States
(12) Patent Application Publication
(10) Pub. No.: US 2008/0262224 A1
(43) Pub. Date: Oct. 23, 2008

(54) Method of Preparation of Benzofuran-2-Carboxylic Acid -Amide

(75) Inventors: William M. Clark, King of Prussia, PA (US); Neil Francis Badham, Malvern, PA (US); Qunying Dai, King of Prussia, PA (US); Ann Marie Eldridge, King of Prussia, PA (US); Hayao Matsuhashi, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION

(21) Appl. No.: 10/597,251

(22) PCT Filed: Jan. 21, 2005
(86) PCT No.: PCT/US05/02121

§ 371 Date: Jul. 18, 2006

Related U.S. Application Data
(60) Provisional application No. 60/538,861, filed on Jan. 23, 2004.

(51) Int. Cl. C07D 223/02; C07D 307/78; C07D 209/04; C07D 213/02

(52) U.S. Cl. 540/597; 549/467; 548/485; 546/294; 540/602; 540/604

(57) ABSTRACT

This invention relates to a method of preparation of benzofuran-2-carboxylic acid {(S)-3-methyl-1-[(4S,7R)-7-methyl-3-oxo-1-(pyridine-2-sulfonyl)-azepan-4-ylcarbamoyl]-butyl}-amide.

### FIELD OF THE INVENTION

This invention relates to a method of preparation of benzofuran-2-carboxylic acid {(S)-3-methyl-1-[(4S,7R)-7-methyl-3-oxo-1-(pyridine-2-sulfonyl)-azepan-4-ylcarbamoyl]-butyl}amide and 3-methyl-N-[(1S)-3-methyl-1-({[(4S,7R)-7-methyl-3-oxo-1-(2-pyridinylsulfonyl) hexahydro-1H-azepin-4-yl]amino}carbonyl)butyl]furo[3,2-b]pyridine-2-carboxamide.

### BACKGROUND OF THE INVENTION

Benzofuran-2-carboxylic acid {(S)-3-methyl-1-[(4S,7R)-7-methyl-3-oxo-1-(pyridine-2-sulfonyl)-azepan-4-ylcarbamoyl]butyl}-amide (Formula I, below) and 3-methyl-N-[(1S)-3-methyl-1-({[(4S,7R)-7-methyl-3-oxo-1-(2-pyridinylsulfonyl) hexahydro-1H-azepin-4-yl]amino}carbonyl)butyl]furo[3,2-b]pyridine-2-carboxamide (Formula II below) are cysteine protease inhibitors disclosed in International Publication WO 01/70232, published on Sep. 27, 2001, which is herein incorporated by reference in its entirety. Such compounds are particularly useful for treating diseases in which cysteine proteases are implicated, especially diseases of excessive bone or cartilage loss, e.g., osteoporosis, periodontitis, and arthritis; and certain parasitic diseases, e.g., malaria.

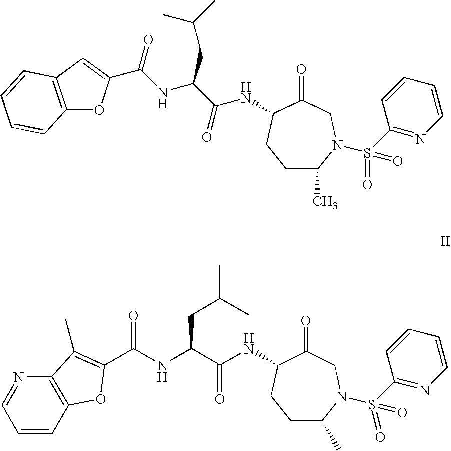

WO 01/70232 also discloses in the Schemes, especially in Schemes 2 and 3, methods of preparation of benzofuran-2-carboxylic acid {(S)-3-methyl-1-[(4S,7R)-7-methyl-3-oxo-1-(pyridine-2-sulfonyl)-azepan-4-ylcarbamoyl]-butyl}amide, 3-methyl-N-[(1S)-3-methyl-1-({[(4S,7R)-7-methyl-3-oxo-1-(2-pyridinylsulfonyl) hexahydro-1H-azepin-4-yl]amino}carbonyl)butyl]furo[3,2-b]pyridine-2-carboxamide and related compounds.

The syntheses disclosed in WO 01/70232 are small-scale multi-step linear processes which utilize a Grubb's cyclization step to construct the core 7-membered azepanone ring system. The starting materials, for instance, Z-D-alaminol, are not necessarily readily available in commercial quantities and in the case of Z-D-alaminol most likely would require expensive custom manufacture to obtain the kilogram amounts required for commercial production of the compound of Formula I and related compounds. Following Grubb's cyclization, the resulting tetrahydroazapene is epoxidized with 3-chloroperoxy benzoic acid (m-CPBA) to give a 3:1 ratio of cis-/trans-epoxides which are separated by chromatography. The isolated epoxides are then taken forward separately. Sodium azide ring opening gives a mixture of regioisomers for one epoxide and only one regioisomer for the other epoxide. Staudinger reduction of the organic azides completes the synthesis of the amino-alcohol diastereomers. The amino-alcohol diasteromers are coupled with Boc-L-leucine and the Cbz protecting group is removed. 2-Chlorosulfonyl pyridine is prepared prior to use from 2-mercaptopyridine/chlorine gas/conc. HCl and is coupled with the free amine to form a sulfonamide. The Boc group is removed with HCl and the amine-HCl is coupled with the desired carboxylic acid, which is 2-benzofuran carboxylic acid when the compound of Formula I is the desired product. A Dess-Martin periodinane oxidation is then employed to furnish the ketone. The α-amino ketone stereocenter is epimerized with Et3N/MeOH heated at reflux to give the correct stereochemistry in the final product.

While useful for the small-scale synthesis of the compounds of Formulas I and II, the methods disclosed in WO 01/70232 are not optimally suited for commercial-scale production. There exists a need for a process suitable to meet the requirements for commercial-scale production of the compounds of Formulas I and II.

### SUMMARY OF THE INVENTION

An object of the present invention is to provide a method for the preparation of benzofuran-2-carboxylic acid {(S)-3-methyl-1-[(4S,7R)-7-methyl-3-oxo-1-(pyridine-2-sulfonyl)-azepan-4-ylcarbamoyl]-butyl}-amide, of Formula I,

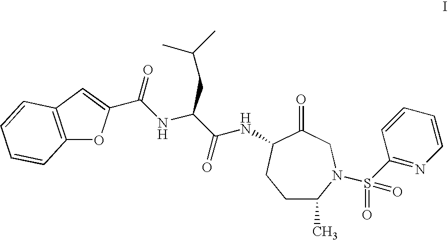

and for the preparation of 3-methyl-N-[(1S)-3-methyl-1-({[(4S,7R)-7-methyl-3-oxo-1-(2-pyridinylsulfonyl) hexahydro-1H-azepin-4-yl]amino}carbonyl)butyl]furo[3,2-b]pyridine-2-carboxamide, of Formula II

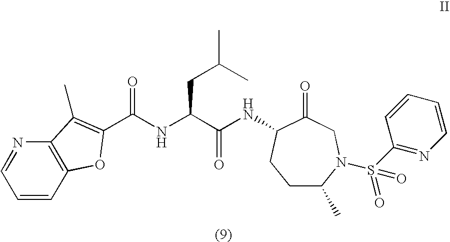

which compounds inhibit cysteine and serine proteases, more particularly cysteine proteases, even more particularly cysteine proteases of the papain superfamily, yet more particularly cysteine proteases of the cathepsin family, most particularly cathepsin K, and which are useful for treating diseases which may be therapeutically modified by altering the activity of such proteases.

Another object of the present invention is to prepare intermediates useful for the preparation of the compounds of Formulas I and II, as well as to provide methods of preparing such intermediates.

### DETAILED DESCRIPTION OF THE INVENTION

The present invention provides a method for the preparation of benzofuran-2-carboxylic acid {(S)-3-methyl-1-[(4S,7R)-7-methyl-3-oxo-1-(pyridine-2-sulfonyl)-azepan-4-ylcarbamoyl]butyl}-amide, of Formula I:

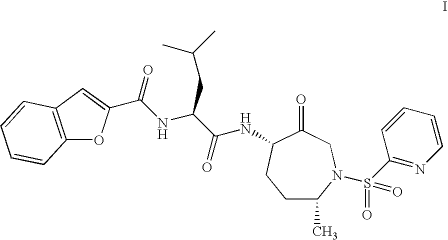

and for the preparation of 3-methyl-N-[(1S)-3-methyl-1-({[(4S,7R)-7-methyl-3-oxo-1-(2-pyridinylsulfonyl) hexahydro-1H-azepin-4-yl]amino}carbonyl)butyl]furo[3,2-b]pyridine-2-carboxamide, of Formula II

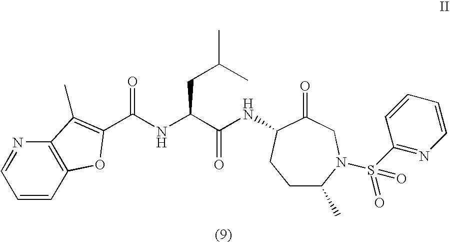

In general, the present invention provides a method, especially suitable for commercial scale manufacture, of preparing the compounds of Formulas I and II. For ease of reference, the compound numbers in the Schemes below are used in the description.

A laboratory-scale method for preparing the compounds of Formulas I and II is disclosed in WO 01/70232. The present method is the most preferred method for preparing the compounds of Formulas I and II on a commercial scale.

The present method comprises the following steps:

A. Preparation of the sulfonamide fragment, further comprising the steps of:

Step 1. Reacting 3-chloro-1-butene 1-1:

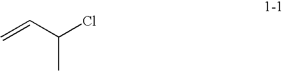

with potassium phthalimide:

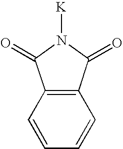

in the presence of an alkali metal carbonate base such as potassium, lithium or sodium carbonate to form compound 1-2 N-(α-methylallyl) phthalimide as a racemate;

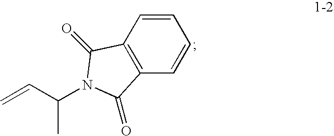

Step 2. chiral chromatography of racemic 1-2 to provide the (R)-enantiomer 1-3, preferably in at least 90% enantiomeric excess. The present invention utilizes a resolution procedure that is conducted by use of chromatography, especially multiple column chromatography (MCC). In contrast to batch chromatography, MCC is a continuous counter-current adsorption process—see U.S. Pat. No. 2,985,589, to Broughton.

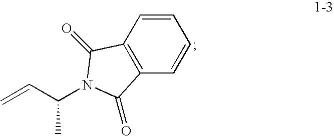

Step 3. Reacting compound 1-3 with a C₁₋₆ alkylamine, C₂₋₆ alkanolamine, or C₂₋₆ alkyldiamine followed by azeotropic distillation with ethanol and gaseous HCl treatment to give compound 1-4, (R)-3-amino-1-butene hydrochloride

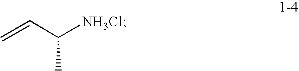

Step 4. 2-chlorosulfonyl pyridine is coupled with the amine hydrochloride 1-4 in the presence of a trialkylamine base to form the pyridine sulfonamide fragment 1-5, (R)-2-pyridinesulfonyl-N-α-methylallyl) amine.

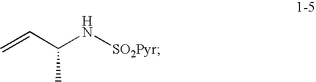

B. Preparation of the epoxide fragment, further comprising the steps of:

Step 1B. epoxidation of 1,4-pentadien-3-ol 2-1,

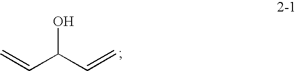

to provide (2S,3R)-1,2-epoxy-4-penten-3ol 2-2

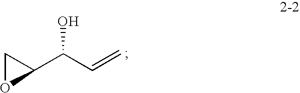

Step 2B. Mitsunobu reaction of epoxide 2-2 to form the nitrogen-protected epoxide fragment 2-3,2-[(1S)-1-(2R)-oxiranyl-2-propenyl]-1H-isoindole-1,3(2H)-dione

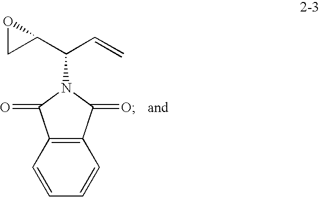

C. Coupling of the sulfonamide fragment and the epoxide fragment further comprising the steps of:

Step 5. Reaction of sulfonamide fragment 1-5 and epoxide fragment 2-3 to provide N-[(2S,3S)-3-(1,3-dihydro-1,3-dioxo-2H-isoindol-2-yl)-2-hydroxy-4-pentenyl]-N-[(1R)-1-methyl-2-propenyl]-2-pyridinesulfonamide 3-1

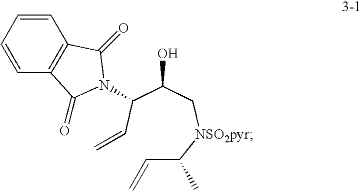

Step 6. Reaction of compound 3-1 with a transition metal alkylidene catalyst, preferably 1,3-bis-2,4,6-trimethylphenyl)-2-imidazolidinylidene) ruthenium (o-isopropoxy-phenylmethylene) dichloride, for the ring closing metathesis to provide compound 3-2, (3S,4S,7R)-4-(1,3-dihydro-1,3-dioxo-2H-isoindol-2-yl)-2,3,4,7-tetrahydro-7-methyl-1-(2-pyridinylsulfonyl)-1H-azepin-3-ol

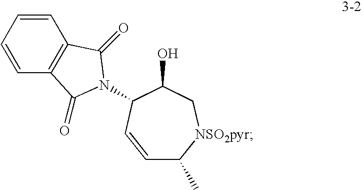

Step 7. Hydrogenation of compound 3-2 to provide the dihydro compound 3-3, (3S,4S,7R)-4-(1,3-dihydro-1,3-dioxo-2H-isoindol-2-yl)-2,3,4,5,6,7-hexahydro-7-methyl-1-(2-pyridinylsulfonyl)-1H-azepin-3-ol

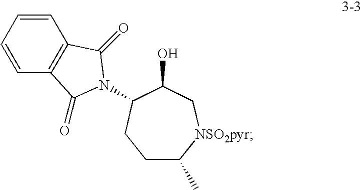

Step 8. Deprotection of the azepanone 4-amino function of compound 3-3 to provide the amino alcohol compound 3-4, (3S,4S,7R)-4-amino-2,3,4,5,6,7-hexahydro-7-methyl-(2-pyridinylsulfonyl)1H-azepin-3-ol that is used in the preparation of compounds of formulas I and II

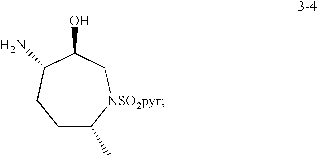

Step 9A (Formula I). Coupling of the amino alcohol 3-4 with the side chain carboxylic acid 3-5, (2S)-2-[(2-benzofuranylcarbonyl)amino]-4-methylpentanoic acid

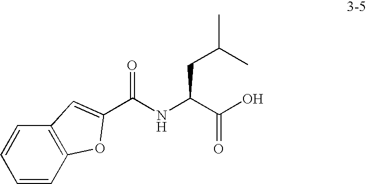

to provide the azepine alcohol 3-6, benzofuran-2-carboxylic acid {(S)-1-[(3S,4S,7R)-3-hydroxy-7-methyl-1-1(pyridine-2-sulfonyl)-azepan-4-ylcarbamoyl]-3-methyl-butyl}-amide

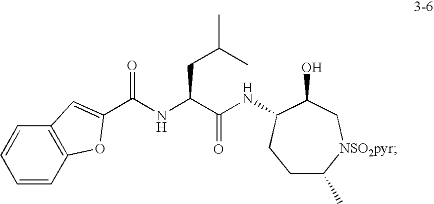

and

Step 10A (Formula I). Oxidation of amino alcohol 3-6 to provide the compound of Formula I.

Or for the compound of Formula II:

Step 9B. (Formula II). Coupling of the amino alcohol 3-4 with the side chain carboxylic acid 6-3 N-[(3-methylfuro[3,2-b]pyridine-2-yl)carbonyl]-L-leucine

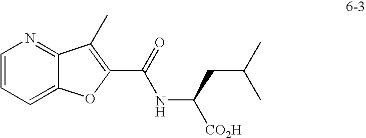

to provide the azepine alcohol 5-1 N-[(1S)-1-({[(3S,4S,7R)-3-hydroxy-7-methyl-1-(2-pyridinylsulfonyl)hexahydro-1H-azepin-4-yl]amino}carbonyl)-3-methylbutyl]-3-methylfuro[3,2-b]pyridine-2-carboxamide

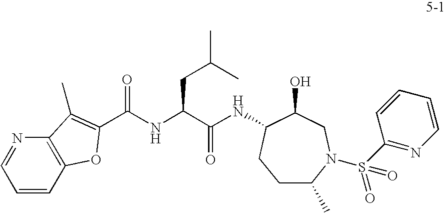

Step 10B (Formula II). Oxidation of amino alcohol 5-1 to provide the compound of Formula II.

The following reaction conditions are useful in the present method:

### A. Preparation of Sulfonamide Fragment

Step 1. This step is conducted in the presence of an alkali metal carbonate base selected from the group consisting of sodium carbonate, lithium carbonate, and potassium carbonate, preferably potassium carbonate, in an aprotic polar solvent, most preferably N,N-dimethylformamide heated at 135° C.

Step 2. The following methods of chiral chromatography selected from the group consisting of: gas chromatography (GC), sub-/supercritical fluid chromatography, high pressure liquid chromatography (HPLC) and multiple column chromatography (MCC) may be used in this step. Preferably, MCC, as described in U.S. Pat. No. 2,985,589 issued to Broughton, using a chiral stationary phase is used to provide compound 1-3 in 80-100% enantiomeric excess, preferably in at least 90%. enantiomeric excess.

Suitably the MCC is carried out in a four zone cascade apparatus which is one of the most efficient implementations of the MCC process—see in U.S. Pat. No. 2,985,589 supra.

The optimal conditions for an MCC separation can be readily identified by analyzing elution profiles obtained from HPLC (high performance liquid chromatography). Important parameters are: loadability of the support, mobile phase strength, selectivity, temperature and feed solubility. The optimization of these parameters aids in identifying conditions for cost-effective separations. The methodology used to identify the conditions for MCC operation is discussed and exemplified in the Journal of Chromatography A, 702 (1995) 97-112.

The preferred MCC procedure may be used as part of a two-stage “enriching-polishing” procedure in which a first pass through MCC is used for enrichment followed by another separation technique to enhance the enrichment. The second stage may be another MCC stage. Alternatively, the second stage may be a different procedure, for example HPLC or crystallization.

Suitable chiral stationary phases for MCC include those sold by Chiral Technologies under the trade mark CHIRALPAK and CHIRALCEL. CHIRALPAK AD, an amylose derivative coated onto silica gel, has been found to be particularly suitable. Other suitable chiral stationary phases (CSPs) are CHIRALCEL OJ, CHIRALCEL OD-H, WHELK-O 1, Kromasil DNB, Kromasil TTB, which are sold by Chiral Technologies, Regis Technologies, and Eka Nobel, respectively.

The mobile phase may be a single component or a mixture of C₅-C₇ alkanes (especially hexane and heptane), C₁-C₃ alkanols (especially methanol, ethanol and 2-propanol), methyl tert-butyl ether (MTBE), ethyl acetate, acetone, and acetonitrile, most preferably ethanol. As used herein, the terms “hexane” and “heptane” refer to straight chain, and branched chain isomers thereof.

Step 3. This step utilizes a C₂₋₆ alkanolamine, C₂₋₆ alkyldiamine, or C₁₋₆ alkylamine which is selected from the group consisting of: OH—(CH₂)ₙ—NH₂, wherein n is 2-6, H₂N—(CH₂)ₙ—NH₂, wherein n is 2-6 and H₃C—(CH₂)ₙ—NH₂, wherein n is 0-5, respectively, more preferably selected from the group consisting of ethanolamine, aminomethane and 1,2-diaminoethane, preferably ethanolamine, and is conducted in an alcoholic solvent, preferably selected from the group consisting of C₁₋₆alkyl alcohols, most preferably ethanol. The amine is purified via azeotropic distillation with ethanol at atmospheric pressure followed by treatment with gaseous HCl to isolate the hydrochloride salt.

Step 4. This step is conducted in the presence of 2-chlorosulfonyl pyridine in an aprotic solvent, e.g., toluene, tetrahydrofuran, ethyl acetate, or methylene chloride, most preferably in methylene chloride, at 25° C., with an amine base such as triethylamine, i-Pr₂EtN, or N-methylmorpholine, most preferably with triethylamine.

### B. Preparation of Epoxide Fragment

Step 1B. This epoxidation may be accomplished using standard Sharpless asymmetric epoxidation conditions. This step is most preferably accomplished in the presence of cumene hydroperoxide or tert-butylhydroperoxide, with Ti(OiPr)₄ and (−)-diisopropyl tartrate ((−)-DIPT) in catalytic or stoichiometric amounts over 4 Å molecular sieves in methylene chloride at −30° C.

Step 2B. The Mitsunobu reaction in this step is most preferably conducted in the presence of phthalimide, triphenylphosphine and diisopropyl azodicarboxylate (DIAD) in an aprotic solvent such as toluene, tetrahydrofuran, ethyl acetate, or methylene chloride, preferably ethyl acetate heated at 20-30° C. Other phthalimides that may be used include succinimide, 4,5-dichlorophthalimide, or 1,8-naphthalimide.

### C. Coupling of Sulfonamide Fragment and Epoxide Fragment

Step 5. Addition of sulfonamide fragment 1-5 and epoxide fragment 2-3 occurs in the presence of a catalytic or stoichiometric amount of a moderately strong amine or phosphazene base such as 1,8-diazabicyclo[5,4,0]-undec-7-ene (DBU), 1,5-diazabicyclo[4.3.0]non-5-ene (DBN), 1,3,4,6,7,8-hexahydro-2H-pyrimido[1,2-a]pyrimidine (TBD), 1,3,4,6,7,8-hexahydro-1-methyl-2H-pyrimido[1,2-a]pyrimidine (MTBD), tert-butylimino-tri(pyrrolidino)phosphorane (BTPP), 1-tert-butyl-2,2,4,4,4-pentakis(dimethylamino)-2λ⁵, 4λ⁵-catenadi(phosphazene) (P2-t-Bu), tert-butylimino-tris(dimethylamino)phosphorane (P1-t-Bu), 1-tert-butyl-4,4,4-tris(dimethylamino)-2,2-bis[tris(dimethylamino)-phosphoranylidenamino]-2%“, 4%”-catenadi(phosphazene) (P4-t-Bu), 1-ethyl-2,2,4,4,4-pentakis(dimethylamino)-2λ⁵, 4λ⁵-catenadi(phosphazene) (P2-Et), preferably tert-butylimino-tri(pyrrolidino)phosphorane (BTPP), in an alcoholic solvent such as isopropanol, ethanol, 2-butanol, 2-pentanol, ethylene glycol, glycerol, or tert-butyl alcohol, most preferably Isopropanol, heated at reflux.

Step 6. This step preferably proceeds in the presence of 1,3-bis-(2,4,6-trimethylphenyl)-2-imidazolidinylidene) ruthenium (o-isopropoxy-phenylmethylene) dichloride in toluene at 110° C. Alternatively, aprotic solvents such as methylene chloride, 1,2-dichloroethane, or tetrahydrofuran (THF) may be also used. The transition metal alkylidene catalysts tricyclohexylphosphine [1,3-bis(2,4,6-trimethylphenyl)-4,5-dihydro-imidazol-2-ylidene][benzylidene]ruthenium (IV) dichloride, bis(tricyclohexylphosphine) benzylidene ruthenium (IV) dichloride, 2,6-diisopropylphenyl-imidoneophylidene molybdenum (VI) bis(hexafluoro-t-butoxide), may alternatively be used in this step.

Step 7. Hydrogenation of compound 3-2 to provide the dihydro compound 3-3 is accomplished using catalytic hydrogenation with a hydrogen pressure of 80-150 psi, preferably 120 psi, over a palladium on carbon catalyst such as PMC catalysts [10% Pd/1625C (wet), 5% Pd/1625C (wet), 10% Pd/2020C (wet), 10% Pd/2055C (wet), 10% Pd/3310C (wet)] in THF or methanol, preferably in THF at 50° C.

Step 8. Deprotection of the azepanone 4-amino function of compound 3-3 occurs in the presence of a suitable amine-substituted compound, preferably methylamine, diaminoethane, or hydrazine monohydrate, most preferably hydrazine monohydrate, in an alcoholic solvent such as methanol or ethanol, preferably ethanol heated at 60° C.

Step 9A (Formula I). Coupling of the amino alcohol 3-4 with the side chain carboxylic acid 3-5 is preferably accomplished in a mixture of 1-[3-(dimethylamino)propyl]-3-ethylcarbodiimide hydrochloride (EDC or EDC.HCl) and 3-hydroxy-1,2,3-benzotriazin-4(3H)-one (HOOBt) in methylene chloride at 0-5° C.

Step 10A (Formula I). Oxidation of azepine alcohol 36 to provide the compound of Formula I preferably occurs in the presence of acetic anhydride in dimethyl sulfoxide at 30-35° C.

### Steps 9B and 10B for the Compound of Formula II:

Step 9B (Formula II). Coupling of the amino alcohol 3-4 with the side chain carboxylic acid 6-3 is preferably accomplished in a mixture of 1-[3-(dimethylamino)propyl]-3-ethylcarbodiimide hydrochloride (EDC or EDC.HCl), 3-hydroxy-1,2,3-benzotriazin-4(3H)-one (HOOBt), and N-methylmorpholine (NMM) in methylene chloride at 0-5° C.

Step 10B (Formula II). Oxidation of azepine alcohol 5-1 to provide the compound of Formula II preferably occurs in the presence of acetic anhydride in dimethyl sulfoxide at 30-35° C.

Schemes 1-6 show the most preferred embodiment of the present invention.

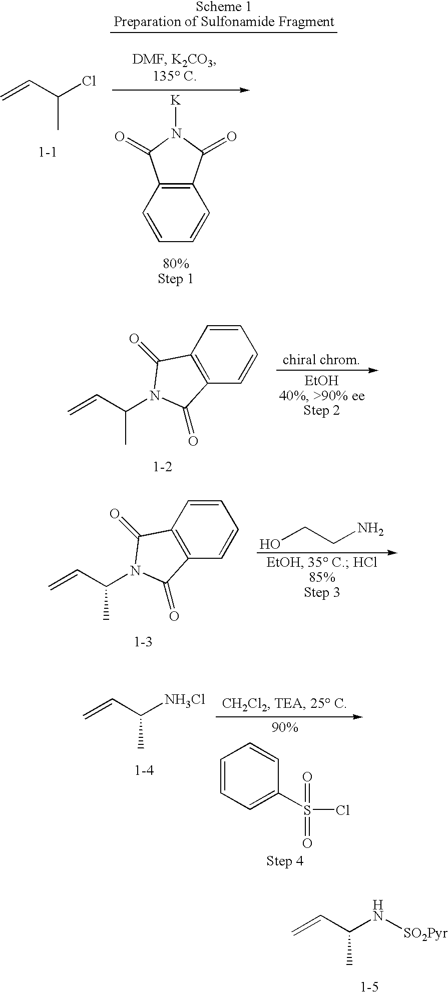

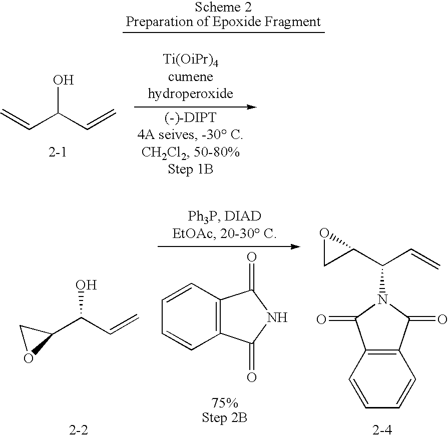

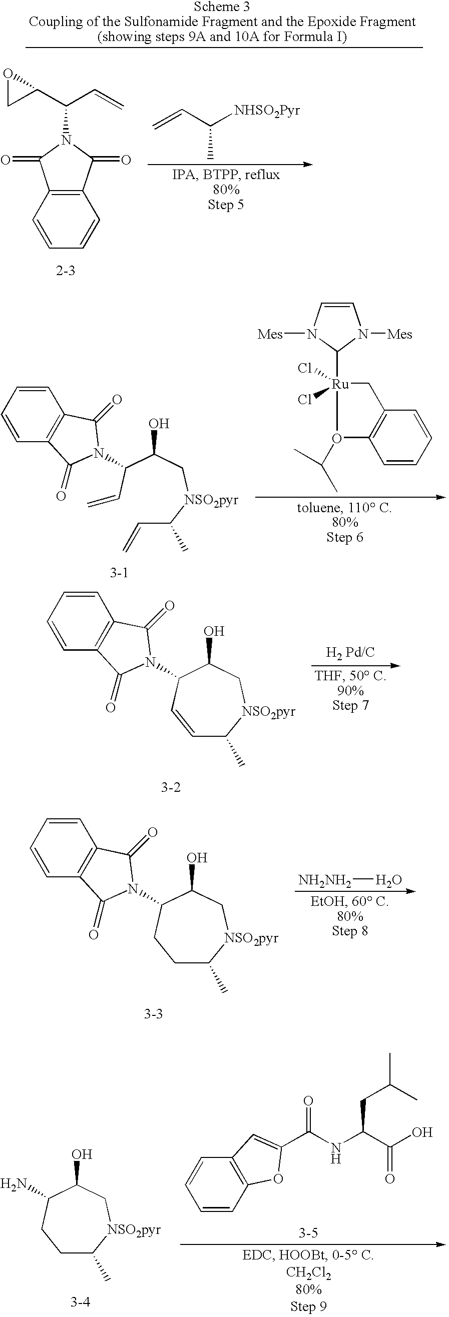

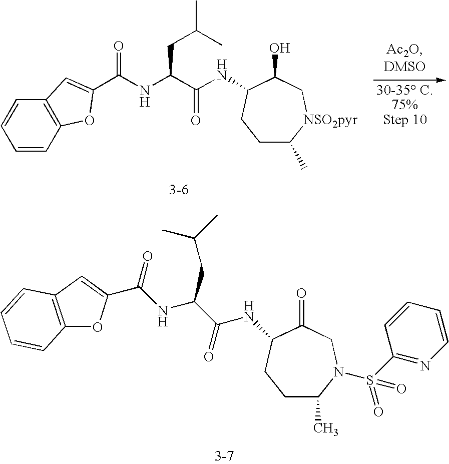

### Scheme 5

### Step 9B and 10B Showing Coupling of the Amino Alcohol with the Side Chain Carboxylic Acid and Oxidation of the Resulting Azepine Alcohol

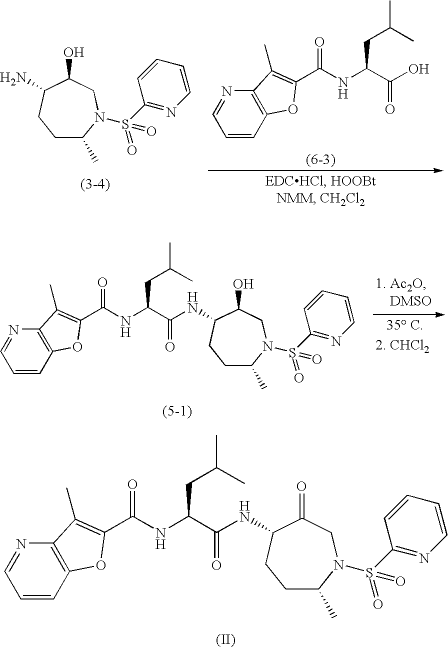

In the most preferred embodiment the following reaction conditions are preferred, as shown in Schemes 1-3, and 6:

### A. Preparation of the Sulfonamide Fragment

Step 1. 3-Chloro-1-butene 1-1 is reacted with potassium phthalimide in the presence of potassium carbonate in dimethylformamide at 135° C. to provide compound 1-2 (R,S)—N-(α-methylallyl) phthalimide. Typically, this step proceeds in 80% by weight yield. This step is described in Semenow, D. et al J. Am. Chem. Soc. 1958, 80, 5472.

Step 2. Chiral chromatography of compound 1-2 to provide the (R)enantiomer 1-3 in at least 90%, preferably greater than 90%, enantiomeric excess, preferably by multiple column chromatography using CHIRALPAK AD as the chiral stationary phase, and ethanol as the mobile phase. Typically, this step proceeds in 40% by weight yield. Multiple column chromatography for the separation of the enantiomers of compound 1-2 is novel.

Step 3. Compound 1-3 is preferably reacted with ethanolamine in ethanol at 35° C. followed by an azeotropic distillation with ethanol at atmospheric pressure of the reaction product 2-amino-3-butene and subsequent treatment of the purified reaction product with gaseous HCl to provide the amine salt 2-amino-3-butene hydrochloride 1-4. Typically, this step proceeds in 85% by weight yield. This step is described in U.S. Pat. No. 4,544,755 to Hagen, et al. The azeotropic distillation of 2-amino-3-butene with ethanol has not been previously reported.

Step 4. 2-Chlorosulfonyl pyridine, in methylene chloride with triethylamine (TEA) at 25° C., is coupled with the amine hydrochloride 1-4 to form a sulfonamide 1-5 (R)-2-pyridinesulfonyl-N-(αmethylallyl) amine. Typically, this step proceeds in 90% by weight yield. This step is described in Goulaouic-Dubois, C. et al Tetrahedron 1995, 51, 12573.

### B. Preparation of the Epoxide Fragment.

Step 1B. Epoxidation of 1,4-pentadien-3-ol 2-1 to provide (2S,3R)-1,2-epoxy-4-penten-3-ol 2-2 preferably occurs in the presence of cumene hydroperoxide, Ti(OiPr)₄ and (−)-DIPT over 4 Å molecular sieves in methylene chloride at −30° C. Typically, this step proceeds in 50-80% by weight yield. This step is described in Romero, A.; Wong, C.-H. J. Org. Chem. 2000, 65, 8264.

Step 2B. The Mitsunobu reaction in this step is preferably conducted in the presence of phthalimide, triphenylphosphine and DIAD in ethyl acetate at 20-30° C. Typically, this step proceeds in 65% by weight yield. This step is described in Kurihara, M., et al., Tetrahedron Lett. 1999, 40, 3183.

### C. Coupling of the Sulfonamide Fragment and the Epoxide Fragment.

Step 5. Addition of sulfonamide fragment 1-5 and epoxide fragment 2-3 occurs in isopropyl alcohol heated at reflux in the presence of tert-butylimino-tri(pyrrolidino)phosphorane (BTPP). Typically, this step proceeds in 80% by weight yield. The addition of a pyridine sulfonamide to an epoxide has not been previously reported.

Step 6. This step preferably proceeds in the presence of 1,3-bis-(2,4,6-trimethylphenyl)-2-imidazolidinylidene) ruthenium (o-isopropoxy-phenylmethylene) dichloride in toluene at 110° C. Typically, this step proceeds in 80% by weight yield. This step is described in Grubbs, R. H.; Scholl, M., et al., J. Am. Chem. Soc. 2000, 122, 3783.

Step 7. Hydrogenation of compound 3-2 to provide the dihydro compound 3-3 is preferably accomplished with hydrogen at 80-120 psi, preferably 80 psi, over a palladium on carbon catalyst such as PMC 10% Pd/1625C (wet) in THF at 50° C. Typically, this step proceeds in 90% by weight yield. This step is described in Sibi, M. P.; Christensen, J. W., J. Org. Chem. 1999, 64, 6434.

Step 8. Deprotection of the azepanone 4-amino function of compound 3-3 preferably occurs in the presence of hydrazine monohydrate in ethanol at 60° C. Typically, this step proceeds in 80% by weight yield. This step is described in Parkes, K. E. B.; Bushnell, D. J. et al., J. Org. Chem. 1994, 59, 3656.

Step 9A. Convergent coupling of the amino alcohol 3-4 with the side chain carboxylic acid 3-5 is preferably accomplished in a mixture of EDC and HOOBt in methylene chloride at 0-5° C. Typically, this step proceeds in 80% by weight yield. This step is described in Koenig, W.; Geiger, R. Chem. Ber. 1970, 103, 2024 and 2034.

Step 10A. Oxidation of azepine alcohol 3-6 to provide the compound of Formula I preferably occurs in the presence of acetic anhydride in dimethylsulfoxide (DMSO) at 30-35° C. Typically, this step proceeds in 75% by weight yield. This step is described in Aibright, J. D.; Goldman, L. J. Am. Chem. Soc. 1965, 87, 4214.

### Or for the Compound of Formula II:

Step 9B. Convergent coupling of the amino alcohol 34 with the side chain carboxylic acid 6-3 is preferably accomplished in a mixture of EDC, HOOBt, and NMM in methylene chloride at 0-5° C. Typically, this step proceeds in 80% by weight yield. This step is described in Koenig, W.; Geiger, R. Chem. Ber. 1970, 103, 2024 and 2034.

Step 10B. Oxidation of azepine alcohol to provide the compound of Formula II preferably occurs in the presence of acetic anhydride in dimethylsulfoxide (DMSO) at 30-35° C. Typically, this step proceeds in 75% by weight yield. This step is described in Albright, J. D.; Goldman, L. J. Am. Chem. Soc. 1965, 87, 4214.

In the present method, 2-chlorosulfonyl pyridine for use in Step 4 is prepared prior to Step 4 from 2-mercaptopyridine and chlorine gas in conc. HCl in Step 1A. The order of execution of Step 1A is not critical so long as it occurs prior to Step 4.

We have further discovered a novel method of preparing the novel side chain carboxylic acid 3-5 used in Step 9A herein above.

In general, but referring to Scheme 4, the side chain carboxylic acid 3-5 is prepared by a method comprising the following steps:

Step 1. Esterification of benzofuran-2-carboxylic acid 4-1 with N-hydroxysuccinimide 4-2 to provide the succinate ester 4-3; and

Step 2. Amidation of succinate ester 4-3 with (L)-leucine 44 to provide the side chain carboxylic acid 3-5.

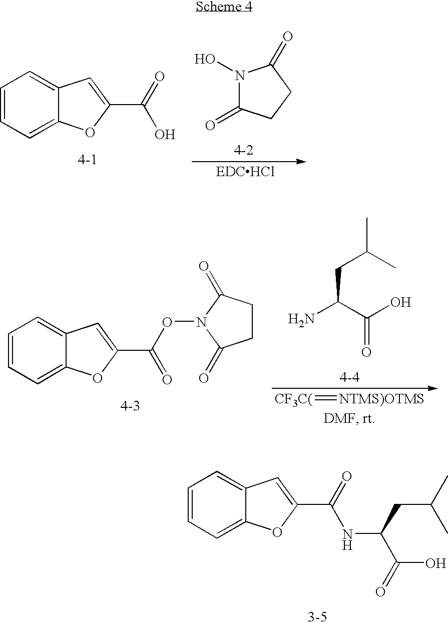

In the most preferred embodiment, shown in Scheme 4, the following reaction conditions are preferred:

Step 1: This step is preferably conducted in the presence of EDC.HCL.

Step 2: This coupling is preferably accomplished using CF₃C(═NTMS)OTMS in dimethyl formamide (DMF) at room temperature.

In general, but referring to Scheme 6, the side chain carboxylic add 6-3 is prepared by a novel method comprising the following steps:

Step 1. esterification of 3-methylfuro[3,2-b]pyridine-2-carboxylic acid 6-1 with N-hydroxysuccinimide 4-2 to provide the succinate ester 6-2; and

Step 2. amidation of succinate ester 6-2 with (L)-leucine 4-4 to provide the side chain carboxylic acid 6-3.

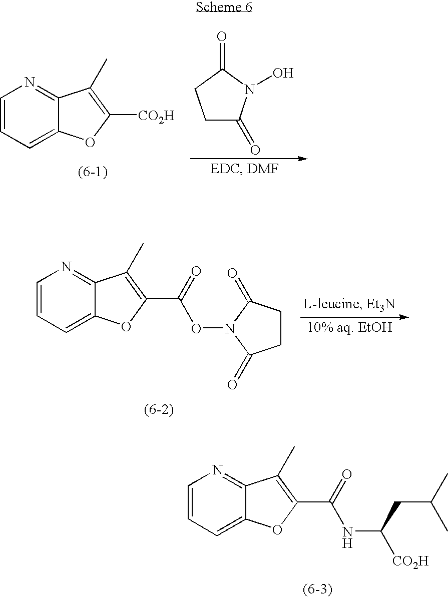

In the most preferred embodiment, shown in Scheme 6, the following reaction conditions are preferred:

Step 1: This step is preferably conducted in the presence of EDC.DMF.

Step 2: This coupling is preferably accomplished using ET₃N in 10% aqueous ethanol at 5-10° C.

The intermediate 6-1 is prepared in the following manner:

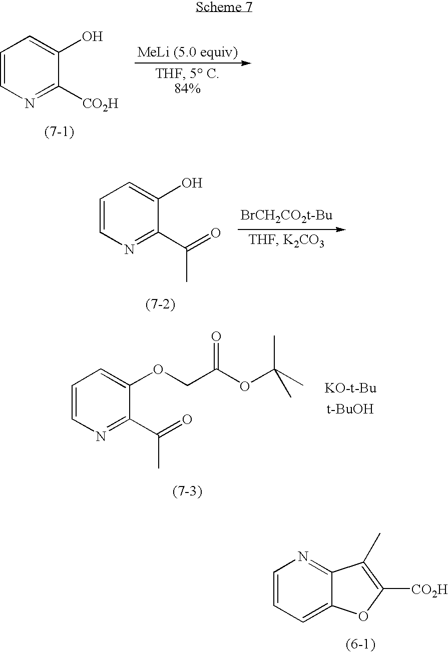

The present method has several advantages, particularly for commercial scale manufacture, over the method disclosed in International Publication WO 01/70232:

1. The present method is a more efficient synthesis for the compound of Formula I providing an improved overall yield;
2. The present method does not use azide chemistry, which is well-known to be hazardous, especially at commercial scales;
3. With elimination of the azide step, the formation of mixtures of the amino alcohols which are generated from opening the epoxide with azide is eliminated. In the present method, the amino alcohol functionality is afforded chirally, producing no mixtures;
4. The stereochemistry of the final product is fixed in step 6, eliminating the ease of handling problems associated with mixtures; and
5. The nitrogen stereochemistry of the final product is set early on in the Mitsunobu reaction in stage 2B, thereby eliminating the need for an equilibration and 15, chromatography step to separate epimers in the final synthetic step.

### Novel Intermediates and Methods of Preparation Thereof

The present invention provides novel intermediates selected from the group consisting of:

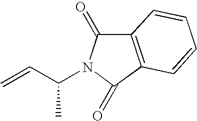

(R)—N-(α-methylallyl) phthalimide, compound 1-3 in Scheme 1:

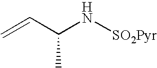

(R)-2-pyridinesulfonyl-N-(α-methylallyl) amine, compound 1-5 in Scheme 1;

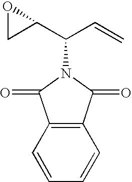

2-[(1S)-1-(2R)-oxiranyl-2-propenyl]-1H-isoindole-1,3(2H)-dione, compound 2-3 in Scheme 2;

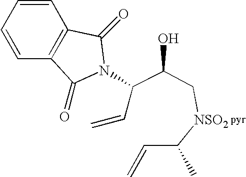

N-[(2S,3S)-3-(1,3-dihydro-1,3-dioxo-2H-isoindol-2-yl)-2-hydroxy-4-pentenyl]-N-[(1R)-1-methyl-2-propenyl]-2-pyridinesulfonamide, compound 3-1 in Scheme 3;

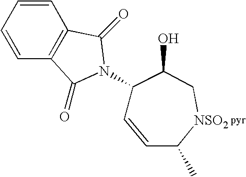

(3S,4S,7R)-(1,3-dihydro-1,3-dioxo-2H-isoindol-2-yl)-2,3,4,7-tetrahydro-7-methyl-1-(2-pyridinylsulfonyl)-1H-azepin-3-ol, compound 3-2 in Scheme 3;

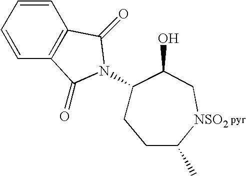

(3S,4S,7R)-4-(1,3-dihydro-1,3-dioxo-2H-isoindol-2-yl)-2,3,4,5,6,7-hexahydro-7-methyl-1-(2-pyridinylsulfonyl)-1H-azepin-3-ol, compound 3-3 in Scheme 3;

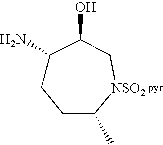

(3S,4S,7R)-4-amino-2,3,4,5,6,7-hexahydro-7-methyl-(2-pyridinylsulfonyl)-1H-azepin-3-ol, compound 3-4 in Scheme 3;

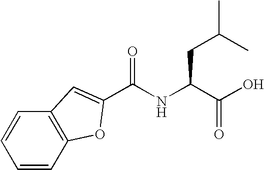

(2S)-2-[(2-benzofuranylcarbonyl)amino]-4-methylpentanoic acid, compound 3-5 in Scheme 3; and

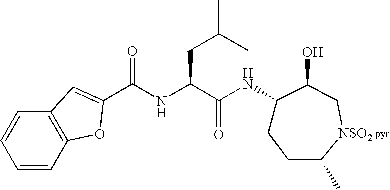

((S)-1-[(3S,4S,7R)-3-hydroxy-7-methyl-1-[(pyridine-2-sulfonyl)-azepan-4-ylcarbamoyl]-3-methyl-butyl]amide, compound 3-6 in Scheme 3.

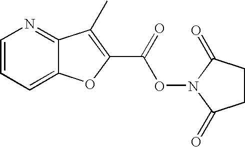

1-({[(4Z)-4-(ethenylimino)-3-methyl-5-methylidene-4,5-dihydro-2-furanyl]carbonyl}oxy)-2,5-pyrrolidinedione compound 6-2 in Scheme 6

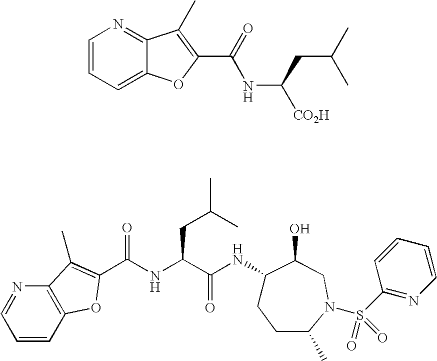

N-[(1S)-1-({[(3S,4S,7R)-3-hydroxy-7-methyl-1-(2-pyridinylsulfonyl)hexahydro-1H-azepin-4-yl]amino}carbonyl)-3-methylbutyl]-3-methylfuro[3,2-b]pyridine-2-carboxamide compound 5-1 in Scheme 5

The sequence of steps in Schemes 1 to 6 and the accompanying description herein above leading to the preparation of any one of the above-identified novel intermediates each constitute a novel method of preparing such novel intermediate.

### Utility of the Present Invention

The present invention provides a method of preparing the compounds of Formulas I and II, which are useful as protease inhibitors, particularly as inhibitors of cysteine and serine proteases, more particularly as inhibitors of cysteine proteases, even more particularly as Inhibitors of cysteine proteases of the papain superfamily, yet more particularly as inhibitors of cysteine proteases of the cathepsin family, most particularly as inhibitors of cathepsin K, in a cost-effective manner at commercial scale.

The compounds of Formulas I and II are particularly useful for treating diseases in which cysteine proteases are implicated, including infections by pneumocystis carinii, trypsanoma cruzi, trypsanoma brucei, and Crithidia fusiculata; as well as in schistosomiasis, malaria, tumor metastasis, metachromatic leukodystrophy, muscular dystrophy, amytrophy; and especially diseases in which cathepsin K is implicated, most particularly diseases of excessive bone or cartilage loss, including osteoporosis, gingival disease including gingivitis and periodontitis, arthritis, more specifically, osteoarthritis and rheumatoid arthritis, Paget's disease; hypercalcemia of malignancy, and metabolic bone disease.

Parasites known to utilize cysteine proteases in their life cycle (and the diseases caused by these parasites) include Trypanosoma cruzi, Trypanosoma Brucei [trypanosomiasis (African sleeping sickness, Chagas disease)], Leishmania mexicana, Leishmania pifanoi, Leishmania major (leishmaniasis), Schistosoma mansoni (schistosomiasis), Onchocerca volvulus [onchocerciasis (river blindness)]Brugia pahangi, Entamoeba histolytica, Giardia lambia, the helminths, Haemonchus contortus and Fasciola hepatica, as well as helminths of the genera Spirometra, Ttichinella, Necator and Ascaris, and protozoa of the genera Cryptospondium, Eimena, Toxoplasma and Naegieria. The compounds of Formulas I and II prepared by the methods of the present invention are suitable for treating diseases caused by these parasites which may be therapeutically modified by altering the activity of cysteine proteases. In particular, such compounds are useful for treating malaria by inhibiting falcipain.

Metastatic neoplastic cells also typically express high levels of proteolytic enzymes that degrade the surrounding matrix, and certain tumors and metastatic neoplasias may be effectively treated with the compounds of Formulas I and II prepared by the methods of this invention.

### General

Nuclear magnetic resonance spectra were recorded at either 300 or 400 MHz using, respectively, a Bruker AM 300 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (δ) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF

instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

### EXAMPLES

In the following example, temperature is in degrees Centigrade (° C.). Unless otherwise indicated, all of the starting materials were obtained from commercial sources. All compound numbers refer to the compounds shown in the Schemes. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### Example 1

### Benzofuran-2-carboxylic acid {(S)-3-methyl-1-[(4S,7R)-7-methyl-3-oxo-1-(pyridine-2-sulfonyl)-azepan-4-ylcarbamoyl]-butyl}-amide

### Preparation of 2-[(1S)-1-(2R)-oxiranyl-2-propenyl]-1H-isoindole-1,3(2H)-dione (Compound 2-3)

To a solution of (2S,3R)-1,2-epoxy-4-penten-3-ol (compound 2-2) (1 g, 9.99 mmol) in ethyl acetate (10 mL) was added triphenylphosphine (3.14 g, 11.97 mmol) and the solution stirred at 20-25° C. until the triphenylphosphine had dissolved. Phthalimide (1.75 g, 11.89 mmol) was then added to this solution. In a separate flask diisopropylazodicarboxylate (DIAD) was dissolved in ethyl acetate (5 mL). The diisopropyl azodicarboxylate solution was added via dropping funnel to the compound 2-2 solution dropwise over about 1 h. During this addition the temperature of the reaction was maintained between 20-30° C. After the addition was complete the solution was concentrated to dryness and reconstituted in toluene. The mixture was cooled to 0-5° C. and held at this temperature for 1 h. The solid was filtered and washed with cold (0-5° C.) toluene (2×3 mL). The organics were combined, tetrabutyl ammonium bromide added (0.1 g), and washed with 20% Na₂CO₃ solution (4 mL) and water (4 mL). Magnesium chloride (0.95 g, 9.99 mmol) and celite (0.95 g) were added. The solution was stirred at 20-25° C. for 12 h then filtered. The filtered solid was washed with toluene (3 mL). The combined organics were concentrated to give an oil. Yield of compound 2-3 for a typical columned reaction is about 75%. ¹H NMR (400 MHz, CDCl₃) δ 7.87 (m, 2H), 7.74 (m, 2H). 6.18 (ddd, J=17.3, 10.4, 7.0 Hz, 1H), 5.33 (ddd, J=17.3, 1.1, 1.1 Hz, 1H), 5.31 (ddd, J=10.4, 1.1, 1.1 Hz, 1H), 4.50 (dddd, J=7.4, 7.0, 1.1, 1.1 Hz, 1H), 3.71 (ddd, J=7.4, 3.9, 2.5 Hz, 1H), 2.95 (dd, J=4.8, 3.9 Hz, 1H), 2.78 (dd, J=4.8, 2.5 Hz, 1H); mp 62.0-64.0° C.

Preparation of N-(α-methylallyl) phthalimide (Compound 1-2. Equipped a 2.0 L 3-necked flask with air-driven mechanical stirrer and thermocouple. Charged the flask with potassium phthalimide (92.6 g, 0.5 mol, 1.0 equiv), and dimethylformamide (760 g, 805 mL). Added anhydrous powdered potassium carbonate (13.8 g, 0.10 mol, 0.20 equiv) followed by 3-chloro-1-butene 1-1 (58.9 g, 65.4 mL, 0.65 moles, 1.30 equiv) and heated the reaction at 132-135° C. for 4.0 h. Concentrated the reaction by distillation under reduced pressure (approximately 120 mL) at 40-50° C. Allowed the reaction to cool to 40° C., then precipitated the product by the addition of water (850 mL) over a period of 15 min at 40° C. Stirred the precipitate at this temperature for 10 min, then slowly cooled the reaction to 20° C. and stirred for 10-15 min. Cooled the reaction to 15° C. and stirred for 5-10 min. Filtered the product and washed with 2-portions of water (100 mL each at 25° C.). Washed with 45/55 ethanol/water (200 mL) which was cooled to 9-13° C. Compound 1-2 was air dried for 2 h then dried in a vacuum oven at 10 mm of Hg at 42° C. under a slow nitrogen flow for 15 h to give a white solid (85 g, 85%). ¹H NMR (300 MHz, CDCl₃) δ 7.83 (dd, 2H), 7.71 (dd, 2H), 6.26-6.14 (m, 1H), 5.27-5.15 (dd, overlapping, 2H), 4.96 (multiplet, 1H), 1.58 (d, 3H, J=7.2); mp 86.0-87.0° C.

Process I for Isolation of (R)—N-(α-methylallyl) phthalimide (Compound 1-3) using multiple column chromatography. Racemic compound 1-2 was fed at a flowrate of 0.6 l/h (total concentration of isomers: 33 g/l of ethanol) into a SMB system comprised of eight columns of 4.8 cm ID by approximately 8 cm length packed with a total of 0.7 kg of CHIRALPAK AD (87.5 g per column). Ethanol was used as the eluent at a flowrate of 4.5 l/h. As a result, an extract was obtained at a flowrate of 4.7 l/h and a raffinate containing compound 1-3 was obtained at a flowrate of 0.5 l/h. The compounds in the raffinate and extract were recovered as white solids after evaporation of the solvent. The performance was determined to be 94.2%-97.1% optical purity and 90% recovery for raffinate. Raffinate (95.5% optical purity): mp 91-93° C.; [α]^{D}₂₅=−39.6° (THF, c=0.866).

Process II for Isolation of (R)—N-(α-methylallyl)phthalimide (Compound 1-3) using multiple column chromatography. Racemic compound 1-2 (300 kg) was fed at a flowrate of 1.25 l/h (total concentration of isomers: 14 g/l of 60/40:EtOH/Heptane into an SMB comprised of eight columns of 20 cm ID by 8.8 cm length packed with a total of 14.4 kg of CHIRALPAK AD (1.8 kg per column). A 60/40 mixture of Ethanol/Heptane was used as the eluant at a flowrate of 6.85 l/h. The separation was conducted at 36-38 bars pressure at a constant temperature of 30 deg C. As a result, an extract was obtained at a flowrate of 6.20 l/h and a raffinate containing compound 1-3 was obtained at a flowrate of 1.90 l/h. The compounds in the raffinate and extract were recovered as white solids after evaporation of the solvent. The performance was determined to be 98.7%-98.8% optical purity and 94.1% recovery for raffinate. Raffinate, 145 kg (98.8% optical purity): mp 91-93° C.; [α]^{D}₂₅=−39.60 (THF, c=0.866). The average production rate was 6.7 kg of feed per day. The corresponding productivity was 0.47 kg of feed/day/kg CSP.

Preparation of (R)-3-Amino-1-butene hydrochloride (Compound 14. Equipped a 250 mL 3-necked flask with air-driven mechanical stirrer and thermocouple. Charged the reactor with an ethanol solution of compound 1-3 from chiral SMB chromatography. The solution contained compound 1-3 (55.14 g, 1 equiv, 0.274 mol) in dry ethanol (114.5 mL). The flask was charged with ethanolamine (70 mL, 4.2 equiv), and the reactor heated to 31-33° C., and stirred under nitrogen for 5-8 h. The reactor was then equipped with a short path distillation apparatus, and the ethanol and free amine (bp 60-61° C.) distilled as an azeotrope at atmospheric pressure. The pot temperature was 90-95° C., and the boiling point of the ethanol+amine distillate was 80-82° C. The weight of the collected fraction was 107 g. Treated the amine/ethanol mixture with dry HCl at 10° C. until the solution gave a pH=1.5-2.5 (by pH paper). Reduced the volume of the solution by 60-65% under reduced pressure and cooled the solution to ambient temperature and added 10 mL of TBME. The solution was added dropwise to a 5° C. solution of TBME (157 g, 212 mL) containing seed crystals (0.05 g) of the amine hydrochloride. Stirred at 5° C. for 15 min and −10° C. for 1 hr. Collected the solid and washed with 12 mL of TBME/ethanol (v/v 95:5) cooled to <5° C. Dried in a vacuum oven to a constant weight at 40° C. for 5 h. A white solid (27.3 g, 92%) was obtained. ¹H NMR (300 MHz, CDCl₃) δ 8.48 (bs, 3H, NH₃⁺), 6.04-5.92 (m, 1H), 5.47 (dd, 1H) 5.35 (dd, 1H), 3.92, (m, 1H), 1.52 (d, 3H, J=6.73 Hz); mp 123.0-125.0° C.

Preparation of (R)-2-Pyridinesulfonyl-N4α-methylallyl) amine (Compound 1-5) A 500 mL 3-necked round bottom flask was equipped with a thermometer and an overhead stirrer. Methylene chloride (50 mL) was charged into the flask followed by compound 1-4 (4.96 g, 46.1 mmol, 1 equiv) and triethylamine (14.1 mL, 10.2 g, 101.4 mmol, 2.2 equiv). The resulting suspension was then cooled to 0° C. in an ice bath. A solution of 2-chlorosulfonyl pyridine (120.1 g, 51.4 mmol, 1.1 equiv, 7.6% w/w) in methylene chloride was then added to the reaction slowly. The reaction was stirred for 3-5 hours at 0-5° C. The reaction mixture was then transferred to a 500 mL separatory funnel and the product was washed with deionized water (100 mL), 10% aqueous citric acid (100 mL), deionized water (100 mL), saturated sodium bicarbonate (100 mL), and deionized water (100 mL). The organic phase was concentrated under reduced pressure to give a white solid (7.44 g, 76%). ¹H NMR (300 MHz, CDCl₃) δ 8.72 (broad d, J=4.2 Hz, 1H), 8.02 (d, J=7.8 Hz, 1H), 7.90 (dd, J=7.7, 1.7 Hz, 1H), 7.49 (dd with some extra coupling, J=7.6, 4.7 Hz, 1H), 5.64 (ddd, J=16.5, 10.4, 6.0 Hz, 1H), 5.08 (dd, J=17.1, 1.1 Hz, 1H), 5.01 (d, J=7.9 Hz, 1H), 4.93 (dd, J=10.3, 1.1 Hz, 1H), 4.05 (m, 1H), 1.22 (d, J=6.8 Hz, 3H); mp 60.0-61.0° C.

Preparation of the Succinate Ester 4-3. Charged a 2 L 3-necked flask equipped with magnetic stirrer bar, thermometer, and nitrogen inlet with benzofuran 2-carboxylic acid (100 g), N-hydroxysuccimide (78.2 g, 1.1 equiv) and N,N-dimethylformamide (620 ml). Stirred and cooled the suspension with an ice bath to 5° C. Added EDC.HCl (142 g, 1.2 equiv) portionwise (3 portions, add every 1 minute). The temperature increased by 20° C. during addition. Removed the ice bath, and stirred the suspension at 25° C. for 9 h. Cooled the solution with an ice bath, and poured it into water (1.5 L). Extracted with ethyl acetate (1.5 L, 500 ml), and washed the combined organic layers with water (2×500 ml) and saturated NaCl aqueous solution (500 ml). Removed the solvent under reduced pressure to afford a yellow solid. Treated the solid with ethyl acetate (40 ml), t-butyl methyl ether (200 ml) and isopropyl ether (200 ml). Filtered under reduced pressure, and washed the solid with iPr₂O (50 ml). Dried the solid under vacuum at 40° C. for 4 h to give a pale yellow solid (136 g, 85%). ¹H NMR (400 MHz, CDCl₃) δ 7.86 (s, 1H), 7.77 (dd, 1H, J=7.9, 0.5 Hz), 7.65 (d, 1H, J=8.5 Hz), 7.56 (td, 1H, J=7.2, 1.2 Hz), 7.39 (dd, 1H, J=7.9, 7.3 Hz), 2.95 (s, 4H).

Preparation I of (2S)-2-[(2-Benzofuranylcarbonyl)amino]-4-methylpentanoic acid (Compound 3-5 Charged a 3 L 3-necked flask equipped with a magnetic stirrer bar, thermometer, 300 ml addition funnel, and nitrogen inlet with (L)-leucine (72.3 g, 1.05 equiv) and DMF (1.0 L). Charged N,O-bistrimethylsilyl trifluoroacetamide (BSTFA) (283 g, 2.1 equiv) into the addition funnel, and added to the reaction suspension at 25° C. for 5 minutes. No significant exothermic reaction occurred during the addition. Stirred the mixture for 3 h by which time the mixture became a clear colorless solution. Added compound 4-3 (136 g, 1 equiv) in one portion at 25° C., and stirred the yellow solution at 25° C. for 112 h. Cooled the solution with an ice bath to 5° C., and poured into 10% citric acid aqueous solution (1.0 L). Extracted with ethyl acetate (2×1.0 L), and washed the combined organic layers with water (3×500 ml) and saturated NaCl aqueous solution (800 ml). Removed the solvent under reduced pressure to afford the solid cake. Added iPr₂O (400 ml) and washed the solid with iPr₂O (200 ml). Dried the solid in vacuo to yield compound 3-5 as off white fine needles (96.5 g, 67%). ¹H NMR (400 MHz, CDCl₃) δ 7.70 (d, 1H, J=7.3 Hz), 7.55 (d, 1H, J=8.4 Hz), 7.54 (s, 1H), 7.46 (m, 1H), 7.33 (td, 1H, J=7.6, 0.8 Hz), 6.98 (brd, 1H, J=8.2 Hz), 4.90 (ddd, 1H, J=8.8, 8.3, 5.0 Hz), 1.85 (m, 3H), 1.04 (d, 3H, J=6.2 Hz); mp 149.0-150.5° C.

Preparation II of (2S)-2-[(2-Benzofuranylcarbonyl)amino]-4-methylpentanoic acid (Compound 3-5). To 1600 L reactor was added water (300 kg) and potassium carbonate (20.2 kg) to give a clear solution while stirring at 22° C. To the reactor was added sodium hydroxide (12.8 kg) to achieve a pH of 12-13 while maintaining the temperature, at 22° C. L-Leucine (35 kg) was then added providing a clear solution followed by the addition of THF (180 kg). The mixture was cooled to 10-15° C. and a solution of benzofuran-2-carbonyl chloride (43.8 kg) in THF (70 kg) added slowly over a 1.5 h period while maintaining the temperature at 10-15° C. The reaction mixture was stirred at 10-15° C. for 30 min. A solution of 35% HCl (43 kg) and deionized water (36 kg) was added portionwise while maintaining the temperature at 10-15° C. to achieve a pH of 2.0. The mixture was stirred for 30 min, toluene (200 kg) added, and stirred for 10 min. After settling, the bottom aqueous layer was removed and the organic layer was washed with deionized water (150 kg). The organic layer was treated with charcoal (2 kg) and the mixture stirred for 30 min. The charcoal was removed by filtration, and THF removed by distillation to collect 250 L of solvent. The mixture was heated to 90° C. to achieve a homogeneous solution and slowly cooled to 5-10° C. with stirring to form a white crystalline suspension. The suspension was stirred for 1 h at 5-10° C., and the solids filtered. The wet cake was re-suspended in cold toluene (5-10° C., 80 kg), the product collected by filtration, and dried at 40° C. under vacuum to give a white solid (52.9 kg, 79%).

Preparation of N-[(2S,3S)-3-(1,3-dihydro-1,3-dioxo-2H-Isoindol-2-yl)-2-hydroxy-4-pentenyl]-N-[(1R)-1-methyl-2-propenyl]-2-pyridinesulfonamide (Compound 3-1) To a 15 L reactor was charged epoxy phthalimide 2-3 (462 g, 2.01 mol), pyridine sulfonamide 1-5 (416 g, 1.96 mol), and 2-propanol (2000 mL) at room temperature. The mixture was stirred at this temperature while tert-butylimino-tri(pyrrolidino)phosphorane (BTPP) (30 ml, 0.098 mol, 5 mol %) was added. The reaction mixture was heated to 80-85° C. for 20 h, followed by cooling to 35° C., and 10% citric acid solution (1000 mL) added. Water (2000 mL) was then added while keeping the temperature in the range of 32-35° C. Seed crystals (1.70 g) were added, and the mixture stirred vigorously for 30 min. Water (4000 mL) was then added over a 30 min period while keeping the temperature in the range of 35-37° C. Upon completion of the water addition, the mixture was cooled to 4° C. and held at this temperature for 10 min. The mixture was filtered and the wet cake washed with water (2000 mL) and cold 2-propanol-water (1:1, 1000 mL, 0-5° C.). The wet cake was dried under vacuum at room temperature to yield SB719145 (710 g, 82.1%). ¹H NMR (300 MHz, CDCl₃) δ 8.63 (m, 1H), 8.04 (d, 1H, J=8.1 Hz), 7.92 (td, J=7.8, 1.8 Hz), 7.85 (m, 2H), 7.72 (m, 2H), 7.49 (ddd, 1H, J=7.5, 4.8, 1.2 Hz), 6.30 (ddd, 1H, J=17.1, 10.2, 8.4 Hz), 5.84 (ddd, 1H, J=17.4, 10.8, 5.1 Hz), 5.39 (d, 1H, J=17.1 Hz), 5.30 (d, 1H, J=10.2 Hz), 5.14 (m, 2H), 4.69 (m, 1H), 4.61 (ddd, 1H, J=9.3, 9.0, 2.7 Hz), 4.41 (m, 1H), 3.57 (dd, 1H, J=15.3, 2.7 Hz), 3.44 (dd, 1H, J=15.0, 9.0 Hz), 1.31 (d, 3H, J=6.9 Hz); mp 95.6-97.5° C.

Preparation of (3S,4S,7R)-4-(1,3-dihydro-1,3-dioxo-2H-isoindol-2-yl)-2,3,4,7-tetrahydro-7-methyl-1-(2-pyridinylsulfonyl)-1H-azepin-3-ol (Compound 3-2) Charge a 2 L 3 necked round bottomed flask with toluene (800 ml, 11 volume) and the diene 3-1 (70.6 g, 160 mmol). With stirring, degas the mixture with vacuum followed by a nitrogen fill at room temperature. Repeat the process three times. Charge the flask with 1,3-bis-(2,4,6-trimethylphenyl)-2-imidazolidinylidene) ruthenium (o-isopropoxy-phenylmethylene) dichloride (502 mg, 0.80 mmol, 0.5 mol %), degas the mixture with vacuum and nitrogen fill at room temperature. Repeat the process two times. Heat the reaction mixture to 110° C. for 5-24 h then cool the mixture to 60° C. Add tetrakis (hydroxymethyl)phosphonium chloride 80% aqueous solution (5.0 ml, 28.0 mmol) and sodium bicarbonate (2.35 g, 28.0 mmol). Stir the mixture vigorously for 12-15 h at 60° C. Cool the mixture to 40° C. and add H₂O (100 ml) and cyclohexane (800 ml). Cool the mixture to room temperature and stir for 30 min. Filter off the solids, and wash the filter cake with water (2×100 mL) and isopropanol (2×50 mL). Dry the solid under vacuum at 35° C. for 24 h to a white solid (56.2 g, 85%). ¹H NMR (300 MHz, CDCl₃) δ 8.89 (m, 1H), 8.13 (d, 1H, J=7.8 Hz), 8.02 (ddd, 1H, J=7.8, 7.8, 1.8 Hz), 7.85 (m, 2H), 7.73 (m, 2H), 7.65 (ddd, 1H, J=7.8, 4.8, 1.2 Hz), 5.61 (m, 1H), 5.25 (m, 2H), 4.69 (m, 1H), 4.49 (dd, 1H. J=9.6, 4.5 Hz), 4.34 (d, 1H, J=16.5 Hz), 3.94 (dd, 1H, J=16.5, 4.5 Hz), 1.39 (d, 3H, J=6.9 Hz); mp 197.0-198.0° C.

Preparation of (3S,4S,7R)-4-(1,3-dihydro-1,3-dioxo-2H-isoindol-2-yl)-2,3,4,5,6,7-hexahydro-7-methyl-1-(2-pyridinylsulfonyl)-1H-azepin-3-ol (Compound 3-3) Tetrahydroazepine 3-2 (11.4 g, 27.6 mmol) and 10% Pd/1625C (5.7 g, 50% water, PMC) were suspended in tetrahydrofuran (150 ml), and the mixture stirred at 60° C. in a pressure vessel under H₂ (100-150 psi) for 3-5 h. The mixture was cooled to room temperature, and filtered through a pad of celite, which was washed thoroughly with dichloromethane. The filtrate was concentrated under reduced pressure to obtain an off white solid which was washed with small amount of ethyl acetate (15 ml), and the solid dried under vacuum to yield compound 3-3 as white fine crystals (9.35 g, 81%). ¹H NMR (300 MHz, CDCl₃) δ 8.90 (m, 1H), 8.10 (d, 1H, J=7.8 Hz), 7.99 (ddd, 1H, J=7.8, 7.5, 1.5 Hz), 7.83 (m, 2H), 7.71 (m, 2H), 7.62 (ddd, J=7.5, 4.8, 1.2 Hz), 4.35 (m, 1H), 4.28 (m, 3H), 3.80 (m, 1H), 2.87 (br, 1H), 2.51 (m, 1H), 1.58 (m, 3H), 1.26 (d, 3H, J=6.9 Hz); 193.0-194.5° C.

Preparation of (3S,4S,7R)/Amino-2,3,4,5,6,7-hexahydro-7-methyl-(2-pyridinylsulfonyl)-1H-azepin-3-ol (Compound 3-4) Perhydroazepine 3-3 (8.93 g, 21.5 mmol) was suspended in ethanol (120 ml, 190 proof) and heated to 50-55° C. Hydrazine monohydrate (5.2 ml, 107 mmol) was added and the suspension heated at 70-75° C. for 1 h. Upon completion, cool the mixture to 35° C. and add methylene chloride (360 mL, 8 vol) to the reaction mixture. Cool the reaction to −5° C., and stir at −5° C. for 10 minutes. Filter off the solids and distill the filtrate under vacuum to minimum volume (90 mL). Add water (27 mL) and methylene chloride (54 mL), and stir vigorously at room temperature for 30 minutes. Allow the layers to settle and remove the aqueous layer. Wash the aqueous layer with methylene chloride (27 mL), combine the organic phase, and concentrate under reduced pressure to obtain compound 3-4 as pale yellow oil (6.40 g, 80%). ¹H NMR (300 MHz, CD₃OD) δ 8.59 (m, 1H), 7.93 (m, 2H), 7.54 (m, 1H), 3.93 (m, 1H), 3.59 (m, 2H), 3.42 (m, 1H), 2.77 (m, 1H), 1.59 (m, 4H), 0.98 (d, 3H, J=6.0 Hz).

### Preparation of benzofuran-2-carboxylic acid {(S)-1-[(3S,4S,7R)-3-hydroxy-7-methyl-1-1(pyridine-2-sulfonyl)-azepan-4-ylcarbamoyl]-3-methyl-butyl}-amide (Compound 3-6)

Charge a 1 L 3-necked flask with amino alcohol 3-4 (30.91 g, 108.31 mmol) in methylene chloride (185 mL) at room temperature. Charge the flask with carboxylic acid 3-5 (29.8 g, 108.31 mmol, 1 equiv) and HOOBt (0.18 g, 1.08 mmol, 0.01 equiv) and cool to 0-5° C. Charge the flask with EDCl (22.8 g, 119.14 mmol, 1.1 equiv) and stir the reaction at 0-5° C. for 5 h. Check the reaction progress with HPLC after 5 hours. Add 3% NaHCO₃ (186 mL) to the reaction, mix well and separate the layers. Collect the bottom methylene chloride layer. Extract the aqueous layer with methylene chloride (31 mL). Combine the organic layers layer and wash with water (186 mL). Concentrate the organic layer to dryness, add toluene (372 mL), heat to 95-100° C., and stir for 5 minutes. Cool the solution to 58° C. over 30 minutes and add seed crystals (10 mg, 0.03% w/w). Stir at 52-55° C. for 30 minutes and add methyl t-butyl ether (310 mL) over 30 minutes at 52-55° C. Cool the suspension to 15° C. over 45 minutes and filter off the solids. Wash the cake with methyl t-butyl ether (186 mL) and dry in an vacuum oven at 42-45° C. for 24 hours to give a white solid (52.7 g, 90%). ¹H NMR (300 MHz, CDCl₃) δ 8.69 (m, 1H), 8.05 (d, 1H, J=8.1 Hz), 7.93 (td, 1H, J=7.8, 1.8 Hz), 7.73 (d, 1H, J=6.9 Hz), 7.51 (m, 3H), 7.43 (ddd, 1H, J=8.4, 6.9, 1.2 Hz), 7.32 (m, 1H), 7.14 (brd, 1H, J=8.4 Hz), 6.92 (br, 1H), 4.67 (m, 1H), 4.19 (dd, 1H, J=16.2, 2.7 Hz), 3.94 (m, 2H), 3.75 (m, 1H), 3.47 (dd, 1H, J=16.2, 1.8 Hz), 3.08 (br, 1H), 1.75 (m, 6H), 1.38 (m, 1H), 1.04 (d, 3H, J=6.6 Hz), 0.99 (t, 6H, J=6.5 Hz).

Preparation of TBME solvate of benzofuran-2-carboxylic acid {(S)-3-methyl-1-[(4S,7R)-7-methyl-3-oxo-1-(pyridine-2-sulfonyl)-azepan-4-ylcarbamoyl]-butyl}-amide (Compound of Formula I). Charge a 500 mL 3-necked flask under nitrogen atmosphere with alcohol 3-6 (20 g, 37 mmol) and DMSO (200 mL, H₂O content <0.01%), and stir at 35° C. until complete dissolution is achieved. Add acetic anhydride (10 mL, 2.88 equiv) in one portion and continue stirring for approximately 15 h at 30-35° C. Cool the reaction to room temperature and dilute with ethyl acetate (200 mL). Add 8% w/v NaHCO₃ solution (120 mL) with controlled addition and stir the mixture at room temperature for 15 min. Allow the layers to settle and remove the bottom aqueous layer. Repeat the 8% w/v NaHCO₃ solution (120 mL) wash, allow layers to separate and remove bottom aqueous layer. Add water (100 mL) to the organic layer, stir for 15 min, allow layers to separate and remove bottom aqueous layer. Concentrate the ethyl acetate layer to dryness. Dissolve residue in EtOAc (60 mL) and add warm TBME (200 mL, 50-55° C.). Seed the solution and stir until a thick suspension has formed. Add warm TBME (200 mL, 50-55° C.) and cool the suspension to −10° C. over 2 h. Stir the suspension at −10° C. for 30 minutes, filter the solid, and wash the filter cake with cold TBME (100 mL, 5° C.) and dry under vacuum to give a white solid (20.9 g, 90%). ¹H NMR (400 MHz, DMSO-d₆) δ 8.73 (ddd, J=0.8, 1.7, 4.7 Hz), 8.59 (d, J=8.4 Hz), 8.23 (d, J=7.1 Hz), 8.09 (ddd, J=1.7, 7.8, 7.8 Hz), 7.98 (ddd, J=0.8, 1.1, 7.8 Hz), 7.77 (dm, J=7.4 Hz), 7.69 (m), 7.67 (dm, J=7.3 Hz), 7.62 (d, J=0.8 Hz), 7.46 (ddd, J=1.3, 7.3, 8.5 Hz), 7.33 (ddd, J=0.8, 7.4, 8.5 Hz), 4.87 (m), 4.59 (m), 4.40 (d, J=18.9 Hz), 4.26 (m), 3.81 (d, J=18.9 Hz), 3.07 (s), 1.92 (m), 1.69 (m), 1.58 (m), 1.10 (s), 0.91 (d, J=6.3 Hz), 0.89 (d, J=6.3 Hz), 0.83 (d, J=6.9 Hz); mp 101-105° C.

Preparation of benzofuran-2-carboxylic acid {(S)-3-methyl-1-[(4S,7R)-7-methyl-3-oxo-1-(pyridine-2-sulfonyl)-azepan-4-ylcarbamoyl]-butyl}-amide (Compound of Formula I). Charge toluene (12 mL) to benzofuran-2-carboxylic acid {(S)-3-methyl-1-[(4S,7R)-7-methyl-3-oxo-1-(pyridine-2-sulfonyl)-azepan-4-ylcarbamoyl]-butyl}-amide (4.23 g) and heat the slurry to 80° C. to dissolve the solids. Cool the solution to 55° C. and add seed material (0.1% by weight). Hold the slurry at 55° C. for one hour then cool slowly to 0° C. Filter and dry the product at 40° C. under vacuum to give a white crystalline solid (3.78 g, 89.4%). ¹H NMR (400 MHz, DMSO-d₆) δ 8.73 (ddd, J=0.8, 1.7, 4.7 Hz), 8.59 (d, J=8.4 Hz), 8.23 (d, J=7.1 Hz), 8.09 (ddd, J=1.7, 7.8, 7.8 Hz), 7.98 (ddd, J=0.8, 1.1, 7.8 Hz), 7.77 (dm, J=7.4 Hz), 7.69 (m), 7.67 (dm, J=7.3 Hz), 7.62 (d, J=0.8 Hz), 7.46 (ddd, J=1.3, 7.3, 8.5 Hz), 7.33 (ddd, J=0.8, 7.4, 8.5 Hz), 4.87 (m), 4.59 (m), 4.40 (d, J=18.9 Hz), 4.26 (m), 3.81 (d, J=18.9 Hz), 1.92 (m), 1.69 (m), 1.58 (m), 0.91 (d, J=6.3 Hz), 0.89 (d, J=6.3 Hz), 0.83 (d, J=6.9 Hz); mp 151.0-153.0° C.

### Example 2

### 3-methyl-N4[(1S)-3-methyl-1-({[(4S,7R)-7-methyl-3-oxo-1-(2-pyridinylsulfonyl) hexahydro-1H-azepin-4-yl]amino}carbonyl)butyl]furo[3,2-b]pyridine-2-carboxamide

### Preparation of 3-methylfuro[3,2-b]pyridine-2-carboxylic acid (6-1)

Under nitrogen, charge the flask with 3 M methyl magnesium chloride in THF (600 mL, 1.80 mol, 5.0 equiv) and THF (500 mL). Heat the solution to 60-65° C. and add a solution of 3-hydroxypicolinic acid (1) (50 g, 0.36 mole), triethylamine (50 mL, 0.36 mole) in THF (250 mL) to the reaction mixture over approximately 3 h. Continue heating at reflux for 2 h. Cool the reaction to 5-10° C. and add methyl formate (44 mL) keeping the reaction temperature less than 20° C. At 10° C., add 10% aqueous HCl to a pH 34. Transfer to separatory funnel and allow the layers to separate. Remove the top organic phase and extract the aqueous phase with THF (250 mL). Combine the organic phases combined and concentrate to 13-15 volumes and add water (170 mL). Continue with the vacuum distillation until all the THF has been removed. Cool the solution to 10° C., stir 0.5 h, filter and dry 1-(3-hydroxypyridin2-yl)ethanone (2) as the tan solid (38.1 g, 77% yield).

Charge the flask with acetone (540 mL, 15 volumes), 1-(3-hydroxypyridin2-yl)ethanone (2) (36.0 g, 0.26 mol), potassium carbonate (72.53 g, 0.53 mol, 2 equiv) and t-butyl bromoacetate (51.26 g, 0.26 mol, 1 equiv). Heat the reaction to reflux and stir at this temperature for 2 h. Cool the reaction mixture to room temperature. Filter the mixture and wash the filter cake with acetone (720 mL). Concentrate the filtrate and add TBME (720 volumes). Add water (250 mL), stir for 10 min and remove the bottom aqueous layer. Concentrate the organic layer to dryness to give 1,1-dimethylethyl[(2-acetyl-3-pyridinyl)oxy]acetate (3) as a tan solid (52.7 g, 80% yield).

Charge the flask under nitrogen with 1,1-dimethylethyl[(2-acetyl-3-pyridinyl)oxy]acetate (3) (51.42 g, 0.20 mol) and t-butanol (1500 mL, 30 volumes). Heat to 30° C. and charge with potassium t-butoxide (45.98 g, 0.41 mol, 2.0 equiv.). Heat the reaction to 60° C. for 1 h. Add 3 N HCl (110 mL) until pH is −3 while cooling the reaction mixture to 40° C. Stir for 30 min at 40° C., cool to room temperature and filter the solid. Slurry the filter cake in deionized water (250 mL) at room temperature. Filter the solid, wash the filter cake with deionized water (50 mL) and dry in a vacuum oven at ˜40° C. to give 3-methylfuro[3,2-b]pyridine-2-carboxylic acid (4) as a tan solid (28.07 g, 77% yield).

### Preparation of 1-({[(4Z)-4(ethenylimino)-3-methyl-5-methylidene-4,5-dihydro-2-furanyl]carbonyl}oxy)-2,5-pyrrolidinedione (6-2)

Charge the flask with methylene chloride (268 mL), N-hydroxysuccinimide (19.2 g, 0.167 moles, 1.1 equiv), 1-[3-(dimethylamino)propyl]-3-ethylcarbodiimide hydrochloride (34.8 g, 0.182 moles, 1.2 equiv) and 3-methylfuro[3,2-b]pyridine-2-carboxylic acid (4) (26.8 g, 0.151 moles). Charge the flask with DMF (16.1 mL, 0.6 volumes). Stir at ambient temperature for 5.5 h. Reduce the reaction volume to a minimum stir volume by vacuum distillation and add deionized water (170 mL). Filter the solid and wash the filter cake with water (115 mL). Dry solid under vacuum at 30° C. for 16 h to afford 1-({[(4Z)-4-(ethenylimino)-3-methyl-5-methylidene-4,5-dihydro-2-furanyl]carbonyl}oxy)-2,5-pyrrolidinedione (5) as a tan solid (35.3 g, 85% yield).

Preparation of N-[(3-methylfuro[3,2-b]pyridine-2-yl)carbonyl]-L-leucine (6-3) Charge 1-({[(4Z)-4(ethenylimino)-3-methyl-5-methylidene-4,5-dihydro-2-furanyl]carbonyl}oxy)-2,5-pyrrolidinedione (5) (34 g, 0.124 mol), L-leucine (16.9 g, 0.129 mol, 1.04 equiv), EtOH (190 proof, 170 mL) and deionized water (68 mL). Cool the reaction mixture to 5-10° C. and add triethylamine (17.3 mL, 0.124 mol, 1 equiv) over 8 minutes. Remove the cooling and stir at ambient temperature for approximately 16 h. Add deionized water (170 mL) and adjust the pH to approximately 3.0 with 10% HCl (40 mL). Stir for 0.5 h and filter the solid. Wash the filter cake with deionized water (100 ml) and dry at 30° C. under vacuum to afford N-[(3-methylfuro[3,2-b]pyridine-2-yl)carbonyl]-L-leucine (6) as a white solid (33.2 g, 92% yield).

### Preparation of N-[(1S)-1-({[(3S,4S,7R)-3-hydroxy-7-methyl-142-pyridinylsulfonyl)hexahydro-1H-azepin-4-yl]amino}carbonyl)-3-methylbutyl]-3-methylfuro[3,2-b]pyridine-2-carboxamide (5-1)

Charge the flask with (3S,4S,7R)-4-amino-7-methyl-1-(2-pyridinylsulfonyl)hexahydro-1H-azepin-3-ol (7) (40.2 g, 141 mmol), N-[(3-methylfuro[3,2-b]pyridine-2-yl)carbonyl]-L-leucine (6) (41.0 g, 141 mmol) and 1,2,3-benzotriazin-4(3H)-one (0.46 g, 2.82 mmol, 2 mol %) Add methylene chloride (205 mL, 5 volumes), and cool the mixture to approximately 5° C. Charge with N-methylmorpholine (15.6 mL, 141 mmol) in one portion. Stir the mixture at this temperature until the reaction becomes a clear solution. Charge with EDC HCl (32.4 g, 169 mmol, 1.2 quiv.) in one portion, and stir the mixture at this temperature for 30 min. Allow the mixture to warm to room temperature for approximately 30 min (17-24° C.) and continue stirring for 4 h. Concentrate the reaction mixture under reduced pressure to minimum volume. Add ethyl acetate (480 mL) and 10% citric acid aqueous solution (240 mL, 6 volumes), stir vigorously, and allow the layers to separate. Drain off aqueous layer and charge H₂O (240 mL). Stir vigorously and allow the layers to separate. Drain off aqueous layer and charge 8% NaHCO₃ aqueous solution (240 mL). Stir vigorously and allow the layers to separate. Drain off aqueous layer and charge H₂O (240 mL). Stir vigorously and allow the layers to separate. Drain off the aqueous layer and concentrate the organic phase to dryness to give N-[(1S)-1-({[(3S,4S,7R)-3-hydroxy-7-methyl-1-(2-pyridinylsulfonyl)hexahydro-1H-azepin-4-yl]amino}carbonyl)-3-methylbutyl]-3-methylfuro[3,2-b]pyridine-2-carboxamide (8) as a tan solid (71 g, 90% yield).

### Preparation of 3-methyl-N-[(1S)-3-methyl-1-({[(4S,7R)-7-methyl-3-oxo-1-(2-pyridinylsulfonyl) hexahydro-1H-azepin-4-yl]amino}carbonyl)butyl]furo[3,2-b]pyridine-2-carboxamide (Formula II)

Charge the flask with N-[(1S)-1-({[(3S,4S,7R)-3-hydroxy-7-methyl-1-(2-pyridinylsulfonyl) hexahydro-1H-azepin-4-yl]amino}carbonyl)-3-methylbutyl]-3-methylfuro[3,2-b]pyridine-2-carboxamide (8) (22 g, 39 mmol) and DMSO (220 mL) and heat to 35° C. Add acetic anhydride (11.3 mL, 120 mmol, 3 equiv) in one portion and continue stirring for 12 h. Add 8% w/v NaHCO₃ solution (170 ml) over 20 minutes. After the completion of the addition, allow the layers to settle and remove the aqueous layer. Repeat the 8% w/v NaHCO₃ solution (170 ml) wash, allow layers to settle, and remove the aqueous layer. Add water (220 mL) to the organic layer, allow the layers to settle and remove the aqueous layer. Concentrate the organic layer to dryness and dissolve the residue in chloroform (176 mL). Add t-butyl methyl ether (44 mL) and seed crystals [22 mg, (9)]. Add TBME (154 mL) over 20 min while maintaining the temperature at 45° C. Stir for 1 h, cool to 5° C. and stir for 1 h. Filter the solid and wash the filter cake with TBME (44 mL). Dry the wet cake under high vacuum at 70° C. for >12 h to give 3-methyl-N-[(1S)-3-methyl-1({[(4S,7R)-7-methyl-3-oxo-1-(2-pyridinylsulfonyl) hexahydro-1H-azepin-4-yl]amino}carbonyl)butyl]furo[3,2-b]pyridine2-carboxamide (9) (17 g, 80% yield).

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

1. A method of preparing benzofuran-2-carboxylic acid {(S)-3-methyl-1-[(4S,7R)-7-methyl-3-oxo-1-(pyridine-2-sulfonyl)-azepan-4-ylcarbamoyl]-butyl}-amide, of Formula I: 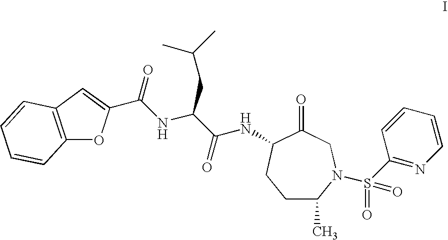
comprising the steps of:
A. preparation of a sulfonamide fragment, further comprising the steps of:
Step 1. reacting 3-chloro-1-butene 1-1: 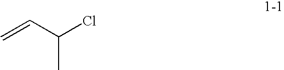
with potassium phthalimide: 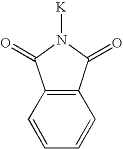
in the presence of an alkali metal carbonate base to form a compound 1-2, N-α-methylallyl) phthalimide, as a racemate; 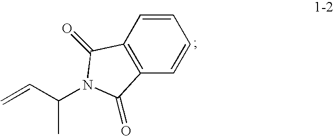
Step 2. chiral chromatography of the racemic compound 1-2 to provide the (R)-enantiomer 1-3; 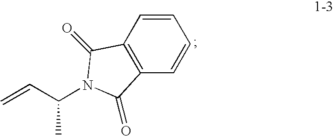
Step 3. reacting the compound 1-3 with a first amine-substituted compound selected from the group consisting of: C₁₋₆ alkylamine, C₂₋₆ alklanolamine, and C₂₋₆ alkyldiamine in an alcoholic solvent to form a reaction product, 2-amino-3-butene, and then purifying the reaction product by azeotropic distillation with ethanol, and then further treating the purified reaction product with gaseous HCl to provide the amine hydrochloride 1-4,2-amino-3-butene hydrochloride 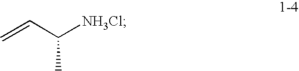
Step 4. Coupling 2-chlorosulfonyl pyridine with the amine hydrochloride 1-4 to form the pyridine sulfonamide fragment 1-5, (R)-2-pyridinesulfonyl-N-α-methylallyl) amine 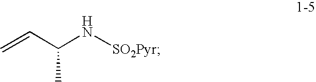
B. preparation of an epoxide fragment, further comprising the steps of:
Step 1B. epoxidation of 1,4-pentadien-3-ol 2-1 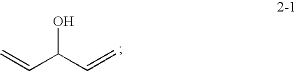
to provide (2S,3R)-1,2-epoxy-4-penten-3-ol 2-2 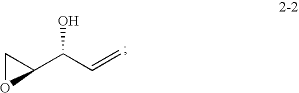
Step 2B. Mitsunobu reaction of (2S,3R)-1,2-epoxy-4-penten-3-ol 2-2 to form the nitrogen protected epoxide fragment 2-3,2-[(1S)-1-(2R)-oxiranyl-2-propenyl]-1H-isoindole-1,3(2H)-dione 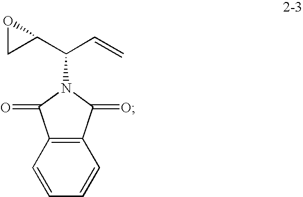
C. coupling of the sulfonamide fragment and the epoxide fragment to provide the compound of Formula I, further comprising the steps of:
Step 5. addition of the sulfonamide fragment 1-5 and the epoxide fragment 2-3 to provide N-(2S,3S)-3-[(1,3-dihydro-1,3-dioxo-2H-isoindol-2-yl)-2-hydroxy-4-pentenyl]-N-[(1R)-1-methyl-2-propenyl]-2-pyridinesulfonamide 3-1 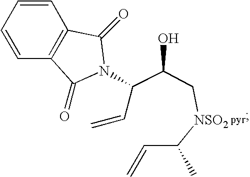
3-1
Step 6. reaction of the compound 3-1 with a transition metal alkylidene catalyst to provide compound 3-2, (3S,4S,7R)-4-(1,3-dihydro-1,3-dioxo-2H-isoindol-2-yl)-2,3,4,7-tetrahydro-7-methyl-1-(2-pyridinylsulfonyl)-1H-azepin-3-ol 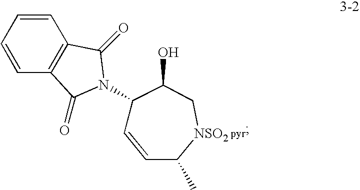
Step 7. hydrogenation of the compound 3-2 to provide the dihydro compound 3-3, (3S,4S,7R)-4-(1,3-dihydro-1,3-dioxo-2H-isoindol-2-yl)-2,3,4,5,6,7-hexahydro-7-methyl-1-(2-pyridinylsulfonyl)-1H-azepin-3-ol 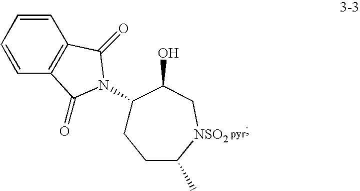
Step 8. deprotection of the azepanone 4-amino function of the compound 3-3 to provide the amino alcohol compound 3-4, (3S,4S,7R)-4-amino-2,3,4,5,6,7-hexahydro-7-methyl-(2-pyridinylsulfonyl)-1H-azepin-3-ol 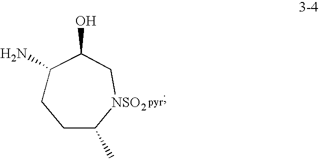
Step 9A. coupling of the amino alcohol 3-4 with the side chain carboxylic acid 3-5, (2S)-2-[(2-benzofuranylcarbonyl)amino]-4-methylpentanoic acid 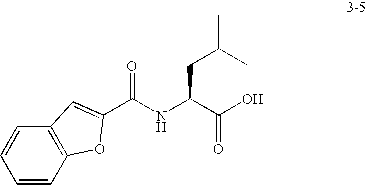
to provide the azepine alcohol 3-6, {(S)-1-[(3S,4S,7R)-3-hydroxy-7-methyl-1-1 (pyridine-2-sulfonyl)-azepan-4-ylcarbamoyl]-3-methyl-butyl}-amide 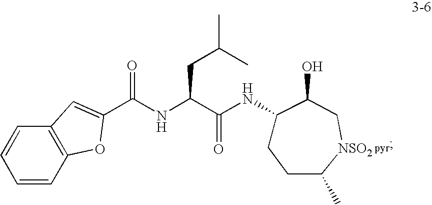
and
Step 10A. oxidation of amino alcohol 3-6 to provide the compound of Formula I.

2. A method of preparing 3-methyl-N-[(1S)-3-methyl-1-({[(4S,7R)-7-methyl-3-oxo-1-(2-pyridinylsulfonyl) hexahydro-1H-azepin-4-yl]amino}carbonyl)butyl]furo[3,2-b]pyridine-2-carboxamide 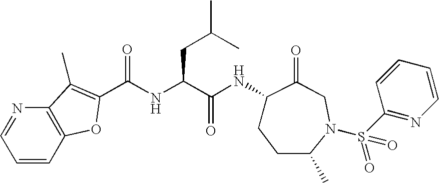
comprising the steps of:
A. preparation of a sulfonamide fragment, further comprising the steps of:
Step 1. reacting 3-chloro-1-butene 1-1: 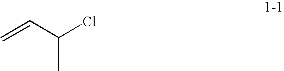
with potassium phthalimide: 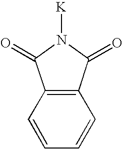
in the presence of an alkali metal carbonate base to form a compound 1-2, N-(α-methylallyl) phthalimide, as a racemate; 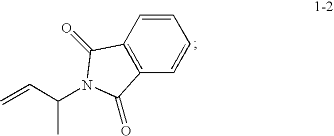
Step 2. chiral chromatography of the racemic compound 1-2 to provide the (R)-enantiomer 1-3; 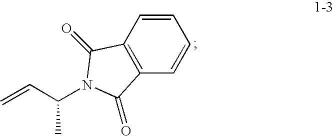
Step 3. reacting the compound 1-3 with a first amine-substituted compound selected from the group consisting of: C₁₋₆ alkylamine, C₂₋₆ alklanolamine, and C₂₋₆ alkyldiamine in an alcoholic solvent to form a reaction product, 2-amino-3-butene, and then purifying the reaction product by azeotropic distillation with ethanol, and then further treating the purified reaction product with gaseous HCl to provide the amine hydrochloride 1-4,2-amino-3-butene hydrochloride 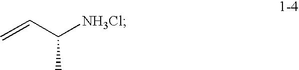
Step 4. Coupling 2-chlorosulfonyl pyridine with the amine hydrochloride 1-4 to form the pyridine sulfonamide fragment 1-5, (R)-2-pyridinesulfonyl-N-(α-methylallyl) amine 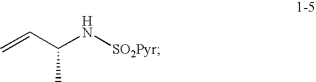
B. preparation of an epoxide fragment, further comprising the steps of:
Step 1B. epoxidation of 1,4-pentadien-3-ol 2-1 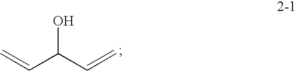
to provide (2S,3R)-1,2-epoxy-4-penten-3-ol 2-2 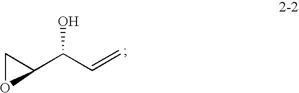
Step 2B. Mitsunobu reaction of (2S,3R)-1,2-epoxy-4-penten-3-ol 2-2 to form the nitrogen protected epoxide fragment 2-3,2-[(1S)-1-(2R)-oxiranyl-2-propenyl]-1H-isoindole-1,3(2H)-dione 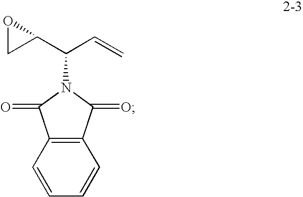
C. coupling of the sulfonamide fragment and the epoxide fragment to provide the compound of Formula I, further comprising the steps of:
Step 5. addition of the sulfonamide fragment 1-5 and the epoxide fragment 2-3 to provide N-(2S,3S)-3-[(1,3-dihydro-1,3-dioxo-2H-isoindol-2-yl)-2-hydroxy-4-pentenyl]-N—[(1R)-1-methyl-2-propenyl]-2-pyridinesulfonamide 3-1 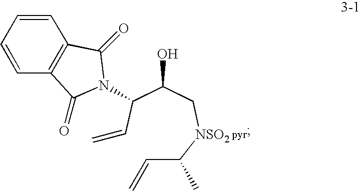
Step 6. reaction of the compound 3-1 with a transition metal alkylidene catalyst to provide compound 3-2, (3S,4S,7R)-4-(1,3-dihydro-1,3-dioxo-2H-isoindol-2-yl)-2,3,4,7-tetrahydro-7-methyl-1-(2-pyridinylsulfonyl)-1H-azepin-3-ol 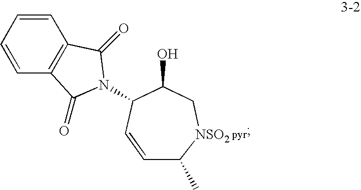
Step 7. hydrogenation of the compound 3-2 to provide the dihydro compound 3-3, (3S,4S,7R)-4-(1,3-dihydro-1,3-dioxo-2H-isoindol-2-yl)-2,3,4,5,6,7-hexahydro-7-methyl-1-(2-pyridinylsulfonyl)-1H-azepin-3-ol 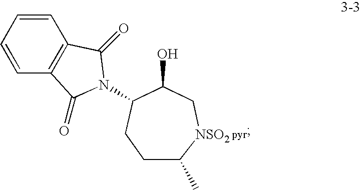
Step 8. deprotection of the azepanone 4-amino function of the compound 3-3 to provide the amino alcohol compound 3-4, (3S,4S,7R)-4-amino-2,3,4,5,6,7-hexahydro-7-methyl-(2-pyridinylsulfonyl)-1H-azepin-3-ol 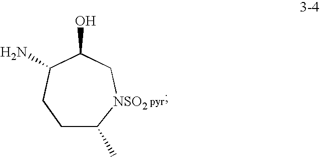
Step 9B. coupling of the amino alcohol 3-4 with the side chain carboxylic acid 6-3, N-[(3-methylfuro[3,2-b]pyridine-2-yl)carbonyl]-L-leucine 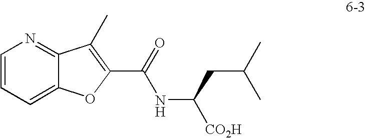
to provide the azepine alcohol 5-1, N-[(1S)-1-({[(3S,4S,7R)-3-hydroxy-7-methyl-1-(2-pyridinylsulfonyl)hexahydro-1H-azepin-4-yl]amino}carbonyl)-3-methyl butyl]-3-methylfuro[3,2-b]pyridine-2-carboxamide 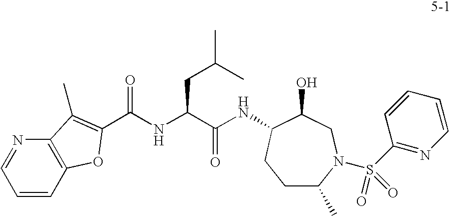
and:
Step 10. Oxidation of amino alcohol 5-1 to provide the compound of Formula II.

3. A method according to claim 1 wherein in Step 1 the alkali metal carbonate base is selected from the group consisting of: sodium carbonate, lithium carbonate, and potassium carbonate and the reaction is carried out in an aprotic polar solvent.

4. A method according to claim 3 wherein the alkali metal carbonate base is potassium carbonate and the aprotic polar solvent is N,N-dimethylformamide which is heated at 135° C.

5. A method according to claim 1 wherein in Step 2 the chiral chromatography is multiple column chromatography where in MCC is used as part of a two-stage “enriching-polishing” procedure wherein in the first stage, a first pass is made using SMB chromatography for enrichment, followed by a second stage wherein a second pass using a second separation technique selected from the group consisting of: MCC, HPLC and crystallization to enhance the enrichment is made.

6. A method according to claim 5 wherein compound 1-3 is provided in 80-100% enantiomeric excess.

7. A method according to claim 5 wherein compound 1-3 is provided in at least 90% enantiomeric excess.

8. A method according to claim 5 wherein the second separation technique is multiple column chromatography.

9. A method according to claim 5 wherein the chiral stationary phase is selected from the group consisting of: CHIRALPAK AD, CHIRALCEL OJ, CHIRALCEL OD-H, WHELK-O 1, Kromasil DNB and Kromasil TTB.

10. A method according to claim 9 wherein the chiral stationary phase is CHIRALPAK AD.

11. A method according to claim 5 wherein the mobile phase is a single component or a mixture selected from the group consisting of: hexane and heptane, methanol, ethanol and 2-propanol, MTBE, ethyl acetate, acetone, and acetonitrile.

12. A method according to claim 1 wherein in Step 3, the C₂₋₆ alkanolamine is ethanolamine, the C₂₋₆ alkyldiamine is 1,2-diaminoethane, and the C₁₋₆ alkylamine is aminomethane, and the alcoholic solvent is ethanol.

13. A method according to claim 1 wherein Step 4 is conducted in an aprotic solvent in the presence of an amine base, wherein the aprotic solvent is selected from the group consisting of: toluene, tetrahydrofuran, ethyl acetate, and methylene chloride and the amine base is selected from the group consisting of: triethylamine, i-Pr₂EtN, and N-methylmorpholine.

14. A method according to claim 13 wherein the aprotic solvent is methylene chloride and the amine base is triethylamine.

15. A method according to claim 1 wherein in Step 1B, the epoxidation is conducted in the presence of a peroxide selected from the group consisting of: cumene hydroperoxide and tert-butylhydroperoxide, with Ti(OiPr)₄ and (−)-diisopropyl tartrate ((−)-DIPT) in catalytic or stoichiometric amounts over 4 Å molecular sieves in methylene chloride at −30° C.

16. A method according to claim 15 wherein the peroxide is cumene hydroperoxide.

17. A method according to claim 1 wherein in Step 2B, the Mitsunobu reaction is conducted in the presence of a phthalimide selected from the group consisting of: phthalimide, succinimide, 4,5-dichlorophthalimide, and 1,8-naphthalimide, triphenylphosphine and diisopropylazodicarbonylate (DIAD) in an aprotic solvent selected from the group consisting of: toluene, tetrahydrofuran, ethyl acetate, and methylene chloride.

18. A method according to claim 17 wherein the phthalimide is phthalimide, and the aprotic solvent is ethyl acetate and the reaction temperature is 20-30° C.

19. A method according to claim 1 wherein in Step 5 the addition of the sulfonamide fragment 1-5 and the epoxide fragment 2-3 occurs in the presence of a catalytic or stoichiometric amount of a moderately strong amine or phosphazene base and in an alcoholic solvent, wherein the moderately strong amine or phosphazene base is selected from the group consisting of: 1,8-diazabicyclo[5,4,0]-undec-7-ene (DBU), 1,5-diazabicyclo[4.3.0]non-5-ene (DBN), 1,3,4,6,7,8-hexahydro-2H-pyrimido[1,2-a]pyrimidine (TBD), 1,3,4,6,7,8-hexahydro-1-methyl-2H-pyrimido[1,2-a]pyrimidine (MTBD), tert-butylimino-tri(pyrrolidino)phosphorane (BTPP), 1-tert-butyl-2,2,4,4,4-pentakis(dimethylamino)-2λ⁵, 4λ⁵-catenadi(phosphazene) (P2-t-Bu), tert-butylimino-tris(dimethylamino)phosphorane (P1-t-Bu), 1-tert-butyl-4,4,4-tris(dimethylamino)-2,2-bis[tris(dimethylamino)-phosphoranylidenamino]-2λ⁵, 4λ⁵-catenadi(phosphazene) (P4-t-Bu), 1-ethyl-2,2,4,4,4-pentakis(dimethylamino)-2λ⁵, 4λ⁵-catenadi(phosphazene) (P2-Et), and the alcoholic solvent is selected from the group consisting of: isopropanol, ethanol, 2-butanol, 2-pentanol, ethylene glycol, glycerol, and tert-butyl alcohol.

20. A method according to claim 19 wherein the moderately strong phosphazene base is tert-butylimino-tri(pyrrolidino)phosphorane (BTPP) and the alcoholic solvent is isopropanol which is at reflux.

21. A method according to claim 1 wherein in Step 6 the reaction occurs in the presence of an aprotic solvent, wherein the aprotic solvent is selected from the group consisting of: 1,2 dichloroethane, methylene chloride, toluene, and tetrahydrofuran (THF) and the transition metal alkylidene catalysts are selected from a group consisting of: 1,3-bis-(2,4,6-trimethylphenyl)-2-imidazolidinylidene) ruthenium (o-isopropoxy-phenylmethylene) dichloride, tricyclohexylphosphine[1,3-bis(2,4,6-trimethylphenyl)-4,5-dihydro-imidazol-2-ylidene][benzylidene]ruthenium (IV) dichloride, bis(tricyclohexylphosphine) benzylidene ruthenium (IV) dichloride, 2,6-diisopropylphenyl-imidoneophylidene molybdenum (VI) bis(hexafluoro-t-butoxide).

22. A method according to claim 21 wherein the aprotic solvent is toluene which is heated to 110° C. and the transition metal alkylidene catalysts is 1,3-bis-(2,4,6-trimethylphenyl)-2-imidazolidinylidene) ruthenium (o-isopropoxy-phenylmethylene) dichloride.

23. A method according to claim 1 wherein in Step 7 the hydrogenation occurs at a hydrogen pressure of 80-150 psi; the hydrogenation catalyst is a palladium on carbon catalyst selected from the group consisting of: 10% Pd/1625C (wet), 5% Pd/1625C (wet), 10% Pd/2020C (wet), 10% Pd/2055C (wet), and 10% Pd/3310C (wet) and the hydrogenation occurs in a solvent selected from the group consisting of: THF and methanol.

24. A method according to claim 23 wherein the hydrogen pressure is 120 psi, and wherein the PMC catalyst is 10% Pd/1625C (wet) and the solvent is THF.

25. A method according to claim 24 wherein the THF is heated at 50° C.

26. A method according to claim 1 wherein Step 8 occurs in the presence of a second amine-substituted compound selected from the group consisting of: methylamine, diaminoethane, and hydrazine monohydrate and wherein Step 8 occurs in an alcoholic solvent selected from the group consisting of: methanol or ethanol.

27. A method according to claim 26 wherein the second amine-substituted compound is hydrazine monohydrate and wherein the alcoholic solvent is ethanol.

28. A method according to claim 27 wherein the ethanol is heated at 60° C.

29. A method according to claim 1 wherein in Step 9A the coupling of the amino alcohol 3-4 with the side chain carboxylic acid 3-5 occurs in a mixture of 1-[3-(dimethylamino)propyl]-3-ethylcarbodiimide hydrochloride (EDC or EDC-HCl) and 3-hydroxy-1,2,3-benzotriazin-4(3H)-one (HOOBt) in methylene chloride at 0-5° C.

30. A method according to claim 1 wherein in Step 10A the oxidation of azepine alcohol 3-6 to provide the compound of Formula I occurs in the presence of acetic anhydride in dimethyl sulfoxide.

31. A method according to claim 30 wherein the dimethyl sulfoxide is heated at 30-35° C.

32. A method of preparation of benzofuran-2-carboxylic acid {(S)-3-methyl-1-[(4S,7R)-7-methyl-3-oxo-1-(pyridine-2-sulfonyl)-azepan-4-ylcarbamoyl]-butyl}-amide, of Formula I: 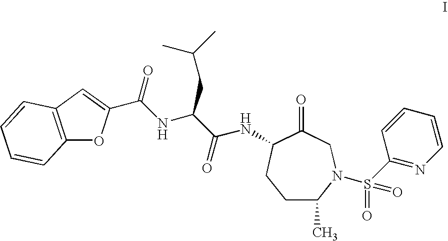
comprising the steps of:
A. preparation of a sulfonamide fragment, further comprising the steps of:
Step 1. reacting 3-chloro-1-butene 1-1 with potassium phthalimide 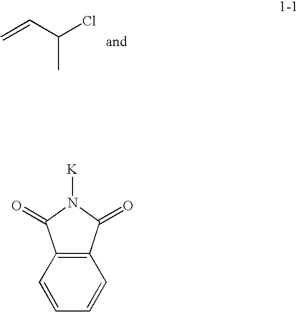
in DMF at 135° C. in the presence of potassium carbonate to form compound 1-2, N-(α-methylallyl) phthalimide as a racemate; 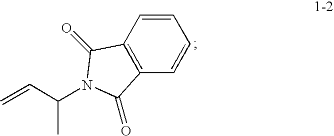
Step 2. Multiple column chromatography of racemic compound 1-2 using CHIRALPAK AD as the chiral stationary phase, and ethanol as the mobile phase, to provide the (R)-enantiomer 1-3 in at least 90% enantiomeric excess 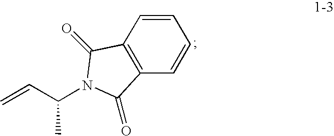
Step 3. reacting compound 1-3 with ethanolamine in ethanol to form a reaction product, 2-amino-3-butene, and then purifying the reaction product by azeotropic distillation with ethanol, and then treating the purified reaction product with gaseous HCl to provide the amine hydrochloride 1-4 2-amino-3-butene hydrochloride 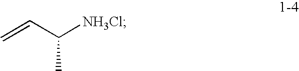
Step 4. Coupling 2-chlorosulfonyl pyridine, in methylene chloride and in the presence of TEA at 25° C., with the amine hydrochloride 1-4 to form the pyridine sulfonamide fragment 1-5, (R)-2-pyridinesulfonyl-N-α-methylallyl) amine 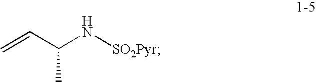
B. preparation of an epoxide fragment, further comprising the steps of:
Step 1B. epoxidation of 1,4-pentadien-3-ol 2-1 in the presence of cumene hydroperoxide, Ti(OiPr)₄ and (−)-DIPT over 4 Å molecular sieves in methylene chloride at −30° C. 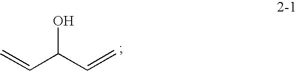
to provide (2S,3R)-1,2-epoxy-4-penten-3-ol 2-2 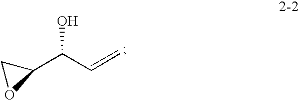
Step 2B. Mitsunobu reaction of the compound 2-2 in the presence of phthalimide, triphenylphosphine and DIAD in toluene at 20-30° C. to form the nitrogen protected epoxide fragment 2-3,2-[(1S)-1-(2R)-oxiranyl-2-propenyl]-1H-isoindole-1,3(2H)-dione 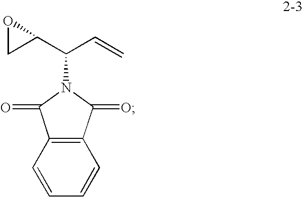
C. coupling of the sulfonamide fragment and the epoxide fragment to provide the compound of Formula I, further comprising the steps of:
Step 5. addition of the sulfonamide fragment 1-5 and the epoxide fragment 2-3 in refluxing isopropyl alcohol in the presence of tert-butylimino-tri(pyrrolidino)phosphorane (BTPP) to provide N-[(2S,3S)-3-(1,3-dihydro-1,3-dioxo-2H-isoindol-2-yl)-2-hydroxy-4-pentenyl]-N-[(1R)-1-methyl-2-propenyl]-2-pyridinesulfonamide 3-1 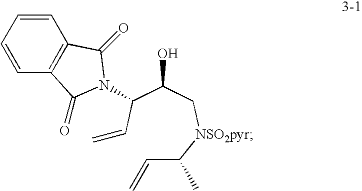
Step 6. reaction of the compound 3-1 with 1,3-bis-(2,4,6-trimethylphenyl)-2-imidazolidinylidene) ruthenium (o-isopropoxy-phenylmethylene) dichloride in toluene at 110° C. to provide the compound 3-2, (3S,4S,7R)-4-(1,3-dihydro-1,3-dioxo-2H-isoindol-2-yl)-2,3,4,7-tetrahydro-7-methyl-1-(2-pyridinylsulfonyl)-1H-azepin-3-ol 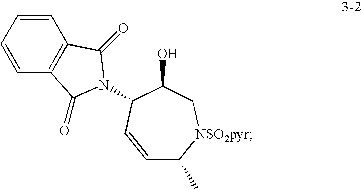
Step 7. catalytic hydrogenation of the compound 3-2 with a hydrogen pressure of 120 psi over PMC 10% Pd/1625C (wet) in THF at 50° C. to provide the dihydro compound 3-3, (3S,4S,7R)-4-(1,3-dihydro-1,3-dioxo-2H-isoindol-2-yl)-2,3,4,5,6,7-hexahydro-7-methyl-1-(2-pyridinylsulfonyl)-1H-azepin-3-ol 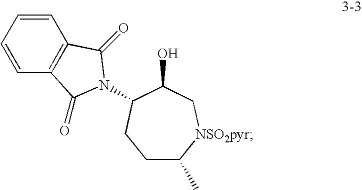
Step 8. deprotection of the azepanone 4-amino function of the compound 3-3 in the presence of hydrazine monohydrate in ethanol at 60° C. to provide the amino alcohol compound 3-4, (3S,4S,7R)-4-amino-2,3,4,5,6,7-hexahydro-7-methyl-(2 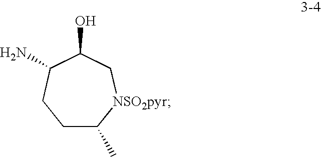
Step 9A. coupling of the amino alcohol 3-4 with the side chain carboxylic acid 3-5 in a mixture of EDC and HOOBt in methylene chloride at 0-5° C. 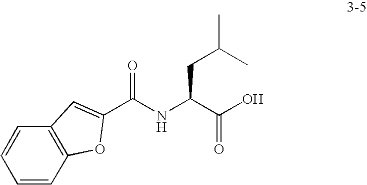
to provide the azepine alcohol 3-6 {(S)-1-[(3S,4S,7R)-3-hydroxy-7-methyl-1-1 (pyridine-2-sulfonyl)-azepan-4-ylcarbamoyl]-3-methyl-butyl}-amide 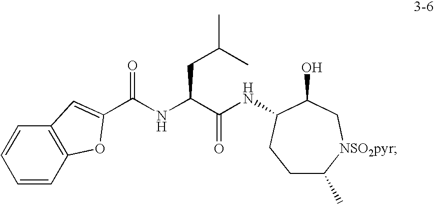
and
Step 10A. oxidation of amino alcohol 3-6 in the presence of acetic anhydride in DMSO at 30-35° C. to provide the compound of Formula I.

33. A method according to claim 32 wherein the 2-chlorosulfonyl pyridine used in Step 4 is prepared before Step 4 by reacting 2-mercaptopyridine, chlorine gas, and conc. HCl.

34. A method for preparing the side chain carboxylic acid 3-5 used in Step 9A of claim 1, comprising the following steps:
Step 1. esterification of benzofuran-2-carboxylic acid 4-1 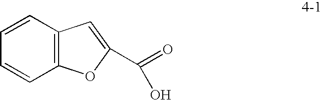
with N-hydroxysuccinimide 4-2 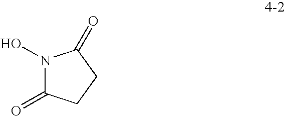
to provide the succinate ester 4-3 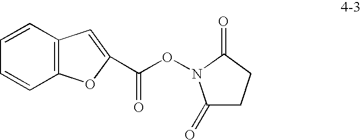
and
Step 2. amidation of succinate ester 4-3 with (L)-leucine 4-4 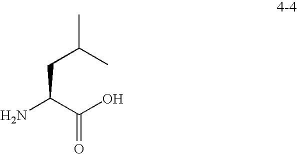
to provide the side chain carboxylic acid 3-5

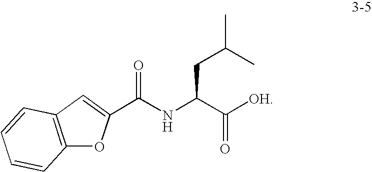

35. A method according to claim 35 wherein Step 1 is conducted in the presence of EDC.HCl and Step 2 is conducted in the presence of CF₃C (═NTMS)OTMS in DMF at room temperature.

36. A method according for preparing the side chain carboxylic acid 3-5 used in Step 9A of claim 1 comprising the following steps:
Step 1. amidation of benzofuran-2-carbonyl chloride 4-5 with (L)-leucine 4-4

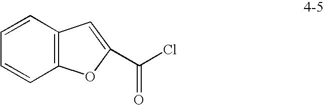

37. A method according to claim 36 wherein Step 1 is conducted in the presence of NaOH and K₂CO₃ in THF at 10-15° C.

38. A method of preparation of 3-methyl-N-[(1S)-3-methyl-1-({[(4S,7R)-7-methyl-3-oxo-1-(2-pyridinylsulfonyl) hexahydro-1H-azepin-4-yl]amino}carbonyl)butyl]furo[3,2-b]pyridine-2-carboxamide, of Formula II: 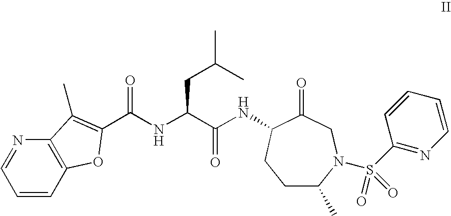
comprising the steps of:
A. preparation of a sulfonamide fragment, further comprising the steps of:
Step 1. Reacting 3-chloro-1-butene 1-1 with potassium phthalimide 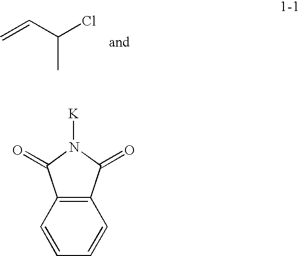
in DMF at 135° C. in the presence of potassium carbonate to form compound 1-2, N-(α-methylallyl) phthalimide as a racemate; 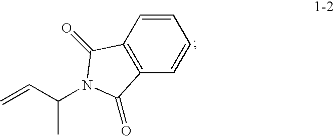
Step 2. Multiple column chromatography of racemic compound 1-2 using CHIRALPAK AD as the chiral stationary phase, and ethanol as the mobile phase, to provide the (R)-enantiomer 1-3 in at least 90% enantiomeric excess 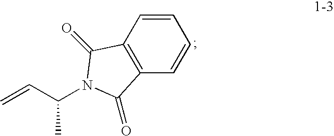
Step 3. Reacting compound 1-3 with ethanolamine in ethanol to form a reaction product, 2-amino-3-butene, and then purifying the reaction product by azeotropic distillation with ethanol, and then treating the purified reaction product with gaseous HCl to provide the amine hydrochloride 1-4 2-amino-3-butene hydrochloride 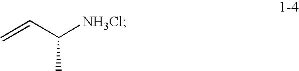
Step 4. Coupling 2-chlorosulfonyl pyridine, in methylene chloride and in the presence of TEA at 25° C., with the amine hydrochloride 1-4 to form the pyridine sulfonamide fragment 1-5, (R)-2-pyridinesulfonyl-N-α-methylallyl) amine 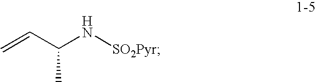
B. preparation of an epoxide fragment, further comprising the steps of:
Step 1B. Epoxidation of 1,4-pentadien-3-ol 2-1 in the presence of cumene hydroperoxide, Ti(OiPr)₄ and (−)-DIPT over 4 Å molecular sieves in methylene chloride at −30° C. 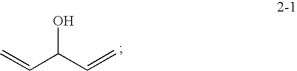
to provide (2S,3R)-1,2-epoxy-4-penten-3-ol 2-2 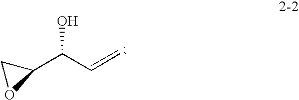
Step 2B. Mitsunobu reaction of the compound 2-2 in the presence of phthalimide, triphenylphosphine and DIAD in toluene at 20-30° C. to form the nitrogen protected epoxide fragment 2-3,2-[(1S)-1-(2R)-oxiranyl-2-propenyl]-1H-isoindole-1,3(2H)-dione 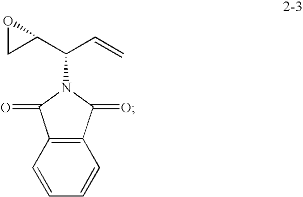
C. coupling of the sulfonamide fragment and the epoxide fragment to provide the compound of Formula I, further comprising the steps of:
Step 5. Addition of the sulfonamide fragment 1-5 and the epoxide fragment 2-3 in refluxing isopropyl alcohol in the presence of tert-butylimino-tri(pyrrolidino)phosphorane (BTPP) to provide N-[(2S,3S)-3-(1,3-dihydro-1,3-dioxo-2H-isoindol-2-yl)-2-hydroxy-4-pentenyl]-N—[(1R)-1-methyl-2-propenyl]-2-pyridinesulfonamide 3-1 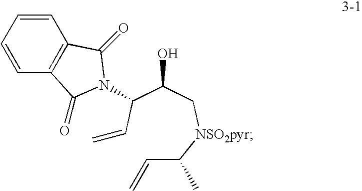
Step 6. Reaction of the compound 3-1 with 1,3-bis-(2,4,6-trimethylphenyl)-2-imidazolidinylidene) ruthenium (o-isopropoxy-phenylmethylene) dichloride in toluene at 110° C. to provide the compound 3-2, (3S,4S,7R)-4-(1,3-dihydro-1,3-dioxo-2H-isoindol-2-yl)-2,3,4,7-tetrahydro-7-methyl-1-(2-pyridinylsulfonyl)-1H-azepin-3-ol 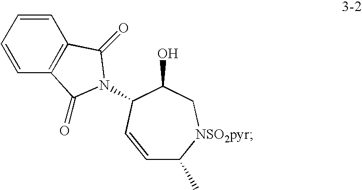
Step 7. Catalytic hydrogenation of the compound 3-2 with a hydrogen pressure of 120 psi over PMC 10% Pd/1625C (wet) in THF at 50° C. to provide the dihydro compound 3-3, (3S,4S,7R)-4-(1,3-dihydro-1,3-dioxo-2H-isoindol-2-yl)-2,3,4,5,6,7-hexahydro-7-methyl-1-(2-pyridinylsulfonyl)-1H-azepin-3-ol 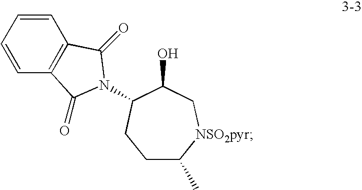
Step 8. Deprotection of the azepanone 4-amino function of the compound 3-3 in the presence of hydrazine monohydrate in ethanol at 60° C. to provide the amino alcohol compound 3-4, (3S,4S,7R)-4-amino-2,3,4,5,6,7-hexahydro-7-methyl-(2 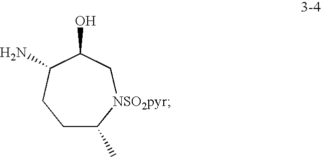
Step 9B: Coupling of the amino alcohol 3-4 with the side chain carboxylic acid 6-3 in a mixture of 1-[3-(dimethylamino)propyl]-3-ethylcarbodiimide hydrochloride (EDC or EDC.HCl), 3-hydroxy-1,2,3-benzotriazin-4(3H)-one (HOOBt), and N-methylmorpholine (NMM) in methylene chloride at 0-5° C. 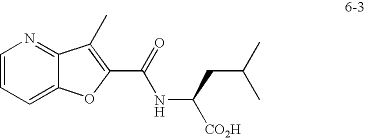
to provide the azepine alcohol 5-1 N-[(1S)-1-({[(3S,4S,7R)-3-hydroxy-7-methyl-1-(2-pyridinylsulfonyl)hexahydro-1H-azepin-4-yl]amino}carbonyl)-3-methyl butyl]-3-methylfuro[3,2-b]pyridine-2-carboxamide; 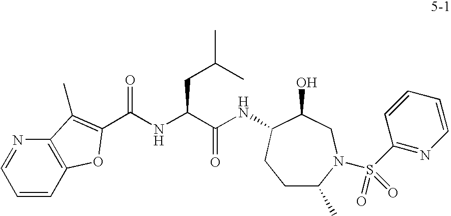
and
Step 10B, oxidation of azepine alcohol 5-1 in the presence of acetic anhydride in dimethyl sulfoxide at 30-35° C. to provide the compound of Formula II.

39. A method according to claim 38 for preparing the side chain carboxylic acid 6-3 used in step 9B, comprising the following steps:
Step 1, Esterification of 3-methylfuro[3,2-b]pyridine-2-carboxylic acid 6-1 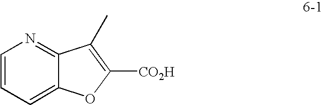
with N-hydroxysuccinimide 4-2 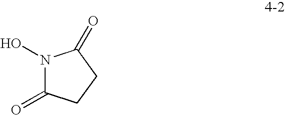
to provide the succinate ester 6-2; 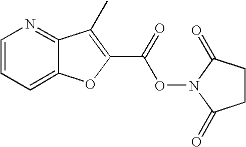
and
Step 2: Amidation of succinate ester 6-2 with (L)-leucine 4-4 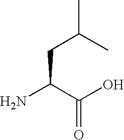
to provide the side chain carboxylic acid 6-3

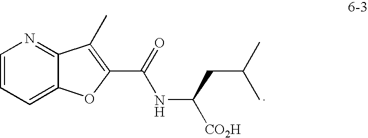

40. A method according to claim 39 wherein step 1 is conducted in the presence of EDC.DMF.

41. A method according to claim 39 wherein step 2 is conducted in the presence of ET₃N in 10% aqueous ethanol at 5-10° C.

42. A compound selected from the group consisting of: 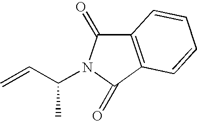
(R)—N-α-methylallyl) phthalimide; 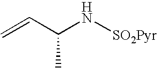
(R)-2-pyridinesulfonyl-N-(□-methylallyl) amine; 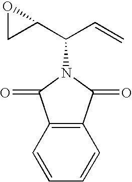
2-[(1S)-1-(2R)-oxiranyl-2-propenyl]-1H-isoindole-1,3(2H)-dione;

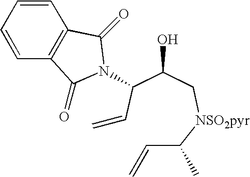

43. A compound selected from the group consisting of:
N-[(2S,3S)-3-(1,3-dihydro-1,3-dioxo-2H-isoindol-2-yl)-2-hydroxy-4-pentenyl]-N-[(1R)-1-methyl-2-propenyl]-2-pyridinesulfonamide; 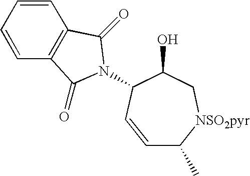
(3S,4S,7R)-4-(1,3-dihydro-1,3-dioxo-2H-isoindol-2-yl)-2,3,4,7-tetrahydro-7-methyl-1-(2-pyridinylsulfonyl)-1H-azepin-3-ol; 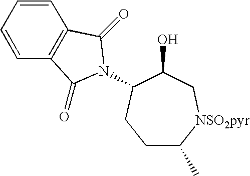
(3S,4S,7R)-4-(1,3-dihydro-1,3-dioxo-2H-isoindol-2-yl)-2,3,4,5,6,7-hexahydro-7-methyl-1-(2-pyridinylsulfonyl)-1H-azepin-3-ol;

44. A compound selected from the group consisting of: 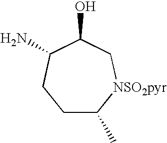
(3S,4S,7R)-4-amino-2,3,4,5,6,7-hexahydro-7-methyl-(2-pyridinylsulfonyl)-1H-azepin-3-ol; 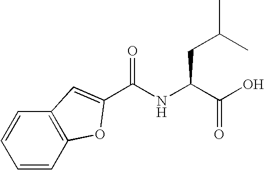
(2S)-2-[(2-benzofuranylcarbonyl)amino]-4-methylpentanoic acid; and 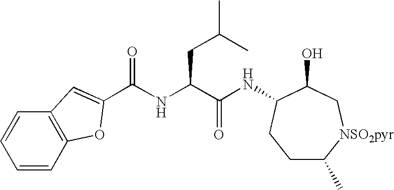
{(S)-1-[(3S,4S,7R)-3-hydroxy-7-methyl-1-1(pyridine-2-sulfonyl)-azepan-4-ylcarbamoyl]-3-methyl-butyl}-amide.
